(19) United States
(12) Patent Application Publication
(10) Pub. No.: US 2026/0294885 A1
(43) Pub. Date: Oct. 1, 2026

(54) TREATMENT OF INFLAMMATORY CONDITIONS COMPRISING IMU-856

(71) Applicant: IMMUNIC AG, Gräfelfing (DE)

(72) Inventors: Franziska BURIANEK, München (DE); Andreas MÜHLER, München (DE); Christian GEGE, Ehingen (DE); Manfred GRÖPPEL, Erlangen (DE); Hella KOHLHOF, München (DE); Daniel VITT, Germering (DE)

(21) Appl. No.: 19/480,481

(22) PCT Filed: May 6, 2024
(86) PCT No.: PCT/EP2024/062464

§ 371 Date: Oct. 31, 2025

(30) Foreign Application Priority Data
May 4, 2023 (EP) 23171616.8

(51) Int. Cl. A61K 31/4439; A61P 1/00; A61P 3/02; A61P 29/00; A61P 37/02

(52) U.S. Cl. CPC A61K 31/4439; A61P 1/00; A61P 3/02; A61P 29/00; A61P 37/02

(57) ABSTRACT

The present invention relates to the sirtuin 6 (SIRT6) modulator IMU-856 or a pharmaceutically acceptable salt or solvate thereof for use in a method of preventing, treating or ameliorating inflammatory conditions, such as gastrointestinal diseases like celiac disease (CelD), comprising administering to a human a therapeutically effective amount of the SIRT6 modulator as well as the corresponding method of preventing, treating or ameliorating inflammatory conditions.

### SUMMARY OF THE INVENTION

The present invention relates to IMU-856 or a pharmaceutically acceptable salt or solvate thereof for use in a method of preventing, treating or ameliorating inflammatory conditions as well as a method of preventing, treating or ameliorating inflammatory conditions, especially gastrointestinal diseases such as celiac disease (CelD), inflammatory bowel disease (IBD) and other diseases that involve a gut barrier defect, by the sirtuin 6 (SIRT6) modulator IMU-856 and/or a pharmaceutically acceptable salt and/or solvate thereof, comprising administering to a human a therapeutically effective amount of the SIRT6 modulator.

### BACKGROUND, INTRODUCTION AND PRIOR ART

Sirtuins are a family of proteins related to the founding member of the family—the silent information regulator 2 protein (Sir2p) of Saccharomyces cerevisiae, a nicotinamide adenine dinucleotide (NAD⁺)-dependent histone deacetylase (HDAC) regulating chromatin silencing. Mammals contain at least seven sirtuin homologues numbered SIRT1 to SIRT7, characterised by significant sequence homology, particularly within their conserved NAD⁺-binding domains (Biochem. Biophys. Res. Commun. 2000; 273: 793). Sirtuins usually possess NAD⁺-dependent deacetylase activity which removes acetyl groups from Ac-Lys of histones or non-histone protein substrates, while producing nicotinamide and acetyl ester metabolites 2′-O- and 3′-O-acetyl-ADP ribose (AADPR). AADPR may themselves function as second messengers in cells. Other sirtuins, e.g., SIRT6 is efficient at deacetylating several histone H3 acetylation sites (J. Am. Chem. Soc. 2023; 12: 6811) and displays a robust NAD⁺-dependent ADP-ribosyl transferase activity (J. Biol. Chem. 2005; 280: 21313). Previous reports suggested that some members of the sirtuin family could negatively regulate inflammatory responses.

WO2008/138943 describes the prophylactic and therapeutic use of sirtuin modulators, and specifically modulators targeting SIRT6, for the treatment of tumor necrosis factor alpha (TNFα)-mediated pathologies, such as various inflammatory and autoimmune disorders (e.g. CelD).

More specific, IMU-856 is a small molecule, orally available and selective inhibitor of the deacetylase activity and stabilizer of the sirtuin 6 protein (SIRT6) covered in WO2019/054427 with chemical formula

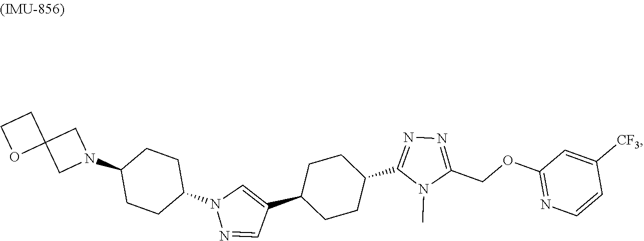 [SMILES: Cn1c(COc2cc(C(F)(F)F)ccn2)nnc1[C@H]1CC[C@H](c2cnn([C@H]3CC[C@H](N4CC5(CCO5)C4)CC3)c2)CC1]

chemical name 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane and with CAS-number 2303528-97-2.

As an orally available and systemically acting small molecule modulator, IMU-856 targets SIRT6, which serves as a transcriptional regulator of intestinal barrier function and regeneration of bowel epithelium. Based on preclinical data, the compound represents a unique treatment approach, as the mechanism of action targets the restoration of the intestinal barrier function and bowel wall architecture in patients suffering from gastrointestinal diseases such as celiac disease, atypical wheat allergy, gluten intolerance (e.g., non-celiac gluten sensitivity (NCGS), gluten ataxia, dermatitis herpetiformis (DH) and wheat allergy), inflammatory bowel disease (IBD) (e.g., Crohn's disease, ulcerative colitis or IBD-associated diarrhea), irritable bowel syndrome (IBS), (especially irritable bowel syndrome with diarrhea (IBS-D)), microscopic colitis (e.g., collagenous colitis and lymphocytic colitis) and eosinophilic esophagitis (EoE).

Targeting SIRT6 with IMU-856 prevents the deacetylation of one of its ligands, acetylated lysine (K) 56 of histone H3 (H3K56), in an in vitro cell free and cellular system and significantly accelerates the recovery of transepithelial electrical resistance (TEER) across a monolayer of colorectal adenocarcinoma-2 (Caco-2) cells following inflammation with tumor necrosis factor (TNF-α) or interleukin (IL)-6. In one TEER model, tight junction (TJ)-related proteins were investigated, and a normalisation of TJ proteins (claudin (Cldn)-1 and claudin-2) was observed. Furthermore, IMU-856 demonstrated efficacy in different settings of the dextran sodium sulphate (DSS)-induced colitis model in mice by reducing disease severity, improving diarrhea symptoms, increasing colon length, and improving colon mucosal histology. IMU-856 mediated inhibition is highly selective for SIRT6 over other members of the human SIRT protein family and does not inhibit enzymatic activity of other histone deacetylases (HDACs).

### BRIEF SUMMARY OF THE INVENTION

The present invention is directed towards IMU-856 or a pharmaceutically acceptable salt or a solvate thereof for use in a method of preventing, treating or ameliorating inflammatory conditions as well as a method of preventing, treating or ameliorating inflammatory conditions, such as gastrointestinal diseases like celiac disease (CelD), or presenting a clinically isolated syndrome, the method comprising administering to the subject or patient IMU-856 or a pharmaceutically acceptable salt or a solvate thereof at a therapeutically effective daily dose of about 34 to about 1370 μmol of IMU-856 or a pharmaceutically acceptable salt or a solvate thereof so as to thereby treat or protect the human patient.

### DETAILED DESCRIPTION OF THE INVENTION

IMU-856 was tested in a first-in-human, three-part, double-blind, randomized, placebo-controlled clinical trial of IMU-856 in healthy subjects and patients with CelD. CelD is a severe autoimmune disorder that occurs in genetically predisposed people and in which the consumption of gluten damages the small intestine. It affects an estimated 1 in 100 people worldwide, but only about 30% are properly diagnosed. When people with celiac disease eat gluten (a protein found in wheat, rye, and barley), their bodies trigger an immune response that attacks the small intestine. These attacks damage the villi, small finger-like projections that line the small intestine and help absorb nutrients. When the villi are damaged, nutrients cannot be properly absorbed by the body. CelD can occur at any age after those affected nave started eating gluten. If left untreated, CelD can lead to other serious health problems.

In Part A, healthy subjects were randomised to single ascending doses of IMU-856 (10 mg to 160 mg) or placebo. In Part B healthy subjects were randomised to multiple once-daily doses of IMU-856 (40, 80, and 160 mg) or placebo for 14 days. Part C randomised patients with celiac disease who were on a gluten-free diet to multiple once-daily doses of IMU-856 (80 mg and 160 mg) or placebo for 28 days; a once-daily gluten challenge (6 g) was introduced on Day 14 and ended on Day 28. 71 healthy subjects were enrolled in Part A and B (N=19 placebo and N=52 IMU-856) and 43 patients with CelD in Part C (N=14 placebo; N=14 for 80 mg, and N=15 for 160 mg). In Parts A and B, treatment-emergent adverse events (TEAEs) occurred in 24 (73%) and 15 (79%) of subjects receiving any dose of IMU-856 compared to 6 (50%) and 5 (71%) with placebo, respectively. TEAEs were mild in severity. In Part C, the most common adverse events with any dose of IMU-856 by preferred term were headache (13 [45%]), nausea (9 [31%]), diarrhoea (8 [28%]), and abdominal distension (7 [24%]). Two serious adverse events occurred with IMU-856 treatment (N=1 Part B and N=1 in Part C), all of which were unrelated to IMU-856. No dose-limiting toxicities, systematic safety laboratory changes or deaths occurred during the study. In Part C, the mean change in villous height from baseline to Day 29 with 80 mg and 160 mg was −20.9±34.8 μm, and −22.5±51.1 μm, respectively, and −60.3±52.2 μm with placebo. Two patients, both treated with IMU-856, showed an improvement in one Q-MARSH category at Day 29, even after gluten challenge. Mean change in plasma citrulline increased with 80 mg and 160 mg from baseline to Day 14 (2.4±6.2 and 6.1±5.9 μmol/L, respectively) and from baseline to Day 29 (2.8±7.6 and 2.2±5.3 μmol/L, respectively), but not with placebo (−2.7±5.6 and −4.0±6.5 μmol/L). Mean increase in vitamin B12 levels from baseline to Day 29 with 80 mg and 160 mg were 45.8±94.4 and 74.2±99.9 μmol/L compared to a mean decrease in placebo (−31.0±68.8 μmol/L). Mean increase in zinc levels from baseline to Day 29 with 80 mg and 160 mg were 0.3±1.4 and 0.9±2.1 μmol/L compared to a mean decrease in placebo (−0.9±2.1 μmol/L). Gluten-induced symptoms were also attenuated by IMU-856. Pharmacokinetics (PK) analysis showed a quick achievement of stable steady-state plasma concentrations within the first week and stable steady-state trough levels over the 14-day treatment period with a low accumulation factor for IMU-856 allowing predictable trough levels during daily dosing. PK parameters in steady-state revealed a tₘₐₓ (time to reach maximum plasma concentration) of 2 to 3 hours post-dose, a plasma half-life of 17.4 to 21.5 hours and dose proportional increases in Cₘₐₓ (maximum plasma drug concentration) and AUC (area under the concentration-time curve).

In light of these findings reported herein, IMU-856 or a pharmaceutically acceptable salt or a solvate, thereof have the potential to be effective in treating CelD with a potentially strong safety and tolerability profile. The obtained results allow the determination of the therapeutically suitable effective daily dose to be from about 34 to about 1370 μmol, preferably about 137 to about 546 μmol, more preferably about 137 to about 273 μmol, of IMU-856 or a pharmaceutically acceptable salt or a solvate thereof. For IMU-856, this translates in an effective daily dose from about 20 mg to about 800 mg, preferably about 80 mg to about 320 mg, more preferably about 60 mg to about 320 mg, even more preferably about 80 mg to about Tou mg, and most preferably about 60 mg to about 240 mg.

In certain embodiments the present invention relates to a method of treating or ameliorating inflammatory conditions, especially gastrointestinal diseases like CelD, as described in the following items. It will be recognized that features specified in each embodiment may be combined with other specified features to provide further embodiments of the present disclosure:

1. A method of preventing, treating or ameliorating a patient afflicted within an flammatory condition am inflammatory condition gut barrier defect, such as gastrointestinal disease, the method comprising administering to the patient IMU-856 and/or a pharmaceutically acceptable salt and/or solvate thereof at a daily dose of about 34 to about 1370 μmol IMU-856 and/or a pharmaceutically acceptable salt and/or solvate thereof, so as to thereby treat the human patient.

2. The method of item 1, wherein the patient is afflicted with a gastrointestinal disease selected from celiac disease, atypical wheat allergy, gluten intolerance (e.g., non-celiac gluten sensitivity, gluten ataxia, dermatitis herpetiformis and wheat allergy), inflammatory bowel disease (e.g., Crohn's disease, ulcerative colitis or inflammatory bowel disease-associated diarrhea), irritable bowel syndrome (especially irritable bowel syndrome with diarrhea), microscopic colitis (e.g., collagenous colitis and lymphocytic colitis), short bowel syndrome and eosinophilic esophagitis.

3. The method according to any one of item 1 to 2, wherein the patient is afflicted with celiac disease.

4. The method according to any one of item 1 to 3, wherein the daily dose is of about 137 to about 411 μmol, preferably of about 137 to about 273 μmol IMU-856 and/or a pharmaceutically acceptable salt and/or solvate thereof.

5. The method according to any one of item 1 to 4, wherein IMU-856 and/or a pharmaceutically acceptable salt and/or solvate thereof is administered orally.

6. The method according to any one of item 1 to 5, wherein IMU-856 and/or a pharmaceutically acceptable salt and/or solvate thereof is administered once or twice daily, preferably once a day.

7. The method according to any one of item 1 to 6, wherein the daily dose of about 80 to 240 mg, preferably of about 80 to about 160 mg IMU-856.

8. The method according to any one of item 1 to 7, wherein IMU-856 and/or a pharmaceutically acceptable salt and/or solvate thereof is administered with at least one additional therapeutic target.

9. The method according to any one of item 1 to 8, wherein treating the gastrointestinal disease resulted in a plasma trough level in the patient, which is above 20 ng/ml of IMU-856.

10. The method according to any one of item 1 to 9, wherein treating the gastrointestinal disease herein comprises favorably altering the level of one or more biomarkers.

11. The method according to any one of item 1 to 10, wherein treating the gastrointestinal disease herein comprises enhancing nutrient absorption.

12. A compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I)

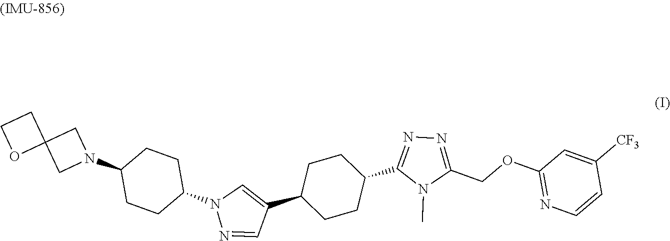 [SMILES: Cn1c(COc2cc(C(F)(F)F)ccn2)nnc1[C@H]1CC[C@H](c2cnn([C@H]3CC[C@H](N4CC5(CCO5)C4)CC3)c2)CC1]

or a pharmaceutically acceptable salt or a solvate thereof for use as a medicament for preventing, ameliorating or treating an inflammatory condition in a patient in need thereof, wherein the compound of formula (I) or a pharmaceutically acceptable salt or solvate thereof is administered to said patient in a daily dose of about 34 to about 1370 μmol.

13. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or a solvate thereof for use as a medicament for preventing, ameliorating or treating an inflammatory condition in a patient in need thereof according to item 12, wherein said inflammatory condition is a gastrointestinal disease.

14. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or a solvate thereof for use as a medicament for preventing, ameliorating or treating an inflammatory condition in a patient in need thereof according to item 13, wherein the gastrointestinal disease selected from celiac disease, atypical wheat allergy, gluten intolerance (e.g., non-celiac gluten sensitivity, gluten ataxia, dermatitis herpetiformis and wheat allergy), inflammatory bowel disease (e.g., Crohn's disease, ulcerative colitis or inflammatory bowel disease-associated diarrhea), irritable bowel syndrome (especially irritable bowel syndrome with diarrhea), microscopic colitis (e.g., collagenous colitis and lymphocytic colitis), short bowel syndrome and eosinophilic esophagitis.

15. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or a solvate thereof for use as a medicament for preventing, ameliorating or treating an inflammatory condition in a patient in need thereof according to item 14, wherein said gastrointestinal disease is a celiac disease.

16. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or a solvate thereof for use as a medicament for preventing, ameliorating or treating an inflammatory condition in a patient in need thereof according to any of items 12 to 15, wherein said daily dose is of about 137 to about 411 μmol, preferably of about 137 to about 273 μmol.

17. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of items 12 to 16, wherein said compound of formula (I) or a pharmaceutically acceptable salt or a solvate thereof is administered orally.

18. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of items 12 to 17, wherein said compound of formula (I) or a pharmaceutically acceptable salt or a solvate thereof is administered once or twice daily, preferably once a day.

19. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of items 12 to 18, wherein the compound of formula (I) is administered in a daily dose of about 80 to 240 mg, preferably of about 80 to about 160 mg.

20. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of items 12 to 19, wherein said compound of formula (I) or a pharmaceutically acceptable salt or a solvate thereof is administered with at least one additional therapeutic target.

21. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of items 13 to 20, wherein treating the gastrointestinal disease resulted in a plasma trough level in the subject or patient, which is above 20 ng/ml of the compound of formula (I).

22. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of items 13 to 21, wherein treating the gastrointestinal disease comprises favorably altering the level of one or more biomarkers.

23. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of items 13 to 22, wherein treating the gastrointestinal disease comprises enhancing nutrient absorption.

In some embodiments the present invention relates to IMU-856 or a pharmaceutically acceptable salt or a solvate thereof for use in a method of treating celiac disease.

In some embodiments the present invention relates to IMU-856 or a pharmaceutically acceptable salt or a solvate thereof for use in a method of treating atypical wheat allergy.

In some embodiments the present invention relates to IMU-856 or a pharmaceutically acceptable salt or a solvate thereof for use in a method of treating gluten intolerance (e.g., non-celiac gluten sensitivity (NCGS), gluten ataxia, dermatitis herpetiformis (DH) and wheat allergy).

In some embodiments the present invention relates to IMU-856 or a pharmaceutically acceptable salt or a solvate thereof for use in a method of treating inflammatory bowel disease.

In some embodiments the present invention relates to IMU-856 or a pharmaceutically acceptable salt or a solvate thereof for use in a method of treating IBD-associated diarrhea.

In some embodiments the present invention relates to IMU-856 or a pharmaceutically acceptable salt or a solvate thereof for use in a method of treating Crohn's disease.

In some embodiments the present invention relates to IMU-856 or a pharmaceutically acceptable salt or a solvate thereof for use in a method of treating steroid-dependent Crohn's disease.

In some embodiments the present invention relates to IMU-856 or a pharmaceutically acceptable salt or a solvate thereof for use in a method of treating ulcerative colitis.

In some embodiments the present invention relates to IMU-856 or a pharmaceutically acceptable salt or a solvate thereof for use in a method of treating steroid-dependent ulcerative colitis.

In some embodiments the present invention relates to IMU-856 or a pharmaceutically acceptable salt or a solvate thereof for use in a method of treating irritable bowel syndrome (especially irritable bowel syndrome with diarrhea).

In some embodiments the present invention relates to IMU-856 or a pharmaceutically acceptable salt or a solvate thereof for use in a method of treating microscopic colitis (e.g., collagenous colitis and lymphocytic colitis)

In some embodiments the present invention relates to IMU-856 or a pharmaceutically acceptable salt or a solvate thereof for use in a method of treating eosinophilic esophagitis.

In some embodiments the present invention relates to IMU-856 or a pharmaceutically acceptable salt or a solvate thereof for use in a method of treating short bowel syndrome.

The present invention relates to IMU-856 or a pharmaceutically acceptable salt or a solvate thereof for use in a method of treating celiac disease as well as refers to a method for treating celiac disease, including subjects or patients on gluten-free diet (GFD), including patients that are significantly symptomatic despite reasonable or substantial compliance with a GFD. In accordance with the invention, patients are treated with IMU-856 or a pharmaceutically acceptable salt or a solvate thereof.

Celiac disease subjects or patients can be identified, diagnosed or confirmed, for example, by measuring the serum levels of anti-endomysial antibody, anti-tissue transglutaminase antibody (anti-tTG), and/or anti-deamidated gliadin peptide (anti-DGP). Celiac patients may also be diagnosed, for example, by small bowel biopsy and/or capsule endoscopy. In addition, patients can be screened for genes encoding the human leukocyte antigens HLA-DQ2 and HLA-DQ8, which are statistically associated with celiac disease. Finally, CelD patients may be assessed via the Q-MARSH Histology Score (preferably having ongoing active (OACD) CelD e.g., a Q-Marsh score of M3a or worse).

In various embodiments, the celiac disease subject or patient may not be reasonably or substantially compliant with a gluten-free diet, meaning that gluten exposure is not merely of an unintentional nature. In accordance with the invention, the celiac symptoms of these patients can be reduced, ameliorated, or prevented, thereby allowing for some GFD noncompliance. In alternative embodiments, the celiac disease patient is reasonably or substantially compliant with a gluten-free diet, meaning that any significant gluten exposure is inadvertent or infrequent. For example, in some embodiments, prior to treatment with IMU-856 or a pharmaceutically acceptable salt or a solvate thereof the celiac disease patient has been on a GFD for at least about 1 week, at least about 2 weeks, at least about 3 weeks, at least about 4 weeks, at least about 1 month, at least about 2 months, at least about 3 months, at least about 4 months, at least about 5 months, or at least about 6 months. In some embodiments, the celiac disease patient has been on a GFD for at least about 1 year, at least about 2 years, at least about 3 years, at least about 4 years, or at least about 5 years. Despite being on a GFD for a length of time, and despite reasonable compliance with the GFD, the patient may still experience celiac symptoms as described herein. IMU-856 or a pharmaceutically acceptable salt or a solvate thereof in conjunction with the GFD can reduce these symptoms, even when these symptoms are significant or substantial, and/or when even non-GI symptoms are experienced or prevalent.

In various embodiments, the celiac disease is non-responsive to GFD. Subjects or patients with non-responsive celiac disease do not exhibit a histological response to a gluten-free diet. Such patients continue to exhibit small-bowel mucosal villous atrophy during a gluten-free diet which is diagnosed by intestinal biopsy (Pulido et al. Can. J. Gastroenterol. Hepatol. 2013; 27:449 and Spatola et al. Aliment Pharmacol. Ther. 2014; 39:407). The most common reason for this lack of recovery despite being on gluten-free diet is continuing gluten ingestion (intentional and unintentional) because gluten is present in many foods and medications or as contaminations. For example, the patient may exhibit a high sensitivity to gluten. Other potential reasons include, for example, the development of other complications such as irritable bowel syndrome, small bowel bacterial overgrowth, other food intolerances such as lactose intolerance, microscopic colitis, Crohn's disease, ulcerative colitis and pancreatic digestive enzyme insufficiency.

Accordingly, in some embodiments, the present invention treats CelD patients who continue to be exposed to gluten, intentionally and/or unintentionally. In some embodiments, the celiac disease patient is non-responsive to a GFD. In some embodiments, the patient is determined to have irritable bowel syndrome, small bowel bacterial overgrowth, or other food intolerances such as lactose intolerance, gastroesophageal reflux, microscopic colitis, Crohn's disease, ulcerative colitis and pancreatic digestive enzyme insufficiency. In further embodiments, the present invention reduces symptoms in celiac disease patients having, for example, extra-intestinal diseases and/or conditions including, but not limited to, dermatitis herpetiformis, diabetes (Type 1 and 2), autoimmune thyroid disease, anemia, dental-enamel hypoplasia/tooth discoloration, osteopenia or osteoporosis, abnormal liver function tests, joint pain and/or join disease, and recurrent miscarriages or fertility problems. These non-GI conditions may be ameliorated, avoided, or managed in part with an IMU-856 regimen as described herein.

In various embodiments, the present invention relates to IMU-856 or a pharmaceutically acceptable salt or a solvate thereof for use in a method of treating celiac disease as well as refers to a method for treating celiac disease patients having refractory celiac disease. Refractory celiac disease is defined by persistent or recurrent malabsorptive symptoms and damaged intestinal architecture despite strict adherence to a gluten-free diet for at least six to twelve months in the absence of other causes of non-responsive celiac disease and overt malignancy (see e.g., Spatola et al. Aliment Pharmacol. Ther. 2014; 39:407, Rubio-Tapia et al. Gut 2010; 59:547). Some of these patients never respond to a gluten-free diet while others initially respond but have a recurrence of symptoms and intestinal inflammation. Most patients with refractory celiac disease have persistent diarrhea, abdominal pain, malabsorption, and involuntary weight loss in addition to vitamin and mineral deficiencies, anemia, fatigue and malaise. Refractory celiac disease is divided into two types. Type I patients exhibit normal T cell population in the intestinal lining and are conventionally treated with aggressive nutritional support as well as pharmacologic therapies including steroids. In contrast, type II patients show abnormal T-cell population in the intestinal lining. These patients have a poor prognosis as they respond poorly to steroid treatment and have a high chance of developing severe complications such as enteropathy-associated T-cell lymphoma (EATL) and ulcerative jejunitis. Accordingly, in some embodiments, the celiac disease is type I refractory celiac disease. In embodiments where the patient has non-responsive or refractory celiac disease, the patient may undergo an adjunct therapy. Exemplary adjunct therapy includes treatment with any of the additional therapeutic agents as described herein. For example, the celiac disease patient may undergo an adjunct therapy involving an anti-inflammatory such as steroid treatment (e.g., prednisone, budesonide, prednisolone, etc.) or NSAID treatment. Alternatively, the celiac disease patient may undergo adjunct therapy with immunosuppressants and other biological modifiers such as azathioprine, cyclosporin, infliximab and alemtuzumab treatment. Alternatively, the celiac patient may undergo therapy with an antibiotic to control bacterial overgrowth in the gastrointestinal tract. Alternatively, or in addition, the patient may undergo treatment with a probiotic.

In various embodiments, the celiac disease patient is experiencing symptoms, despite substantial compliance with GFD or due to non-compliance. While symptoms may be determined by the attending physician through examination/interview of the patient, there are various tools for quantifying or evaluating a patient's symptoms, well-being, and GFD compliance, which may also be employed. These include, but are not limited to, Celiac Disease Patient Reported Outcome (CeD PRO), Gastrointestinal Symptom Rating Scale (GSRS), Celiac Disease Gastrointestinal Symptom Rating Scale (CeD GSRS), Bristol Stool Form Scale (BSFS), General Well-Being Questionnaire, Short Form 12 Health Survey Version 2 (SF12V2), Celiac Disease Quality of Life Questionnaire (CeD-QoL), Clinician Global Assessment of Disease Activity (CGA), Impact of Celiac Disease Symptoms Questionnaire (ICDSQ) or Celiac Disease Symptom Diary (CDSD), the latter two are preferred. Adherence to gluten-free diet may be assessed by, for example, Celiac Dietary Adherence Test (CDAT) and Gluten-Free Diet Compliance Questionnaire (GFDCQ). Accordingly, in some embodiments, the celiac disease patients are experiencing one or more symptoms as measured by one of the scales described herein.

In an embodiment, the celiac disease patient is experiencing one or more symptoms as assessed by the CDSD Version 2.1® (ImmunogenX LLC) at the start of treatment with IMU-856 or a pharmaceutically acceptable salt or a solvate thereof. The CDSD questionnaire was developed to assess symptom severity in clinical trials in subjects with celiac disease. The CDSD includes five symptoms (diarrhea, abdominal pain, bloating, nausea, tiredness) asking participants about the severity of celiac disease symptoms they experience each day. Subjects rate their symptom severity on a 5-point scale (1=none, 2=mild, 3=moderate, 4=severe, 5=very severe). In various embodiments, the celiac disease patient is experiencing one or more significant or severe symptomatic days (based on CDSD) at the start of treatment with IMU-856 or a pharmaceutically acceptable salt or a solvate thereof.

In various embodiments, administration of IMU-856 or a pharmaceutically acceptable salt or a solvate thereof effectively improves the symptoms and sense of well-being of celiac disease patients. Improvements can be assessed using the various scales as described herein or be determined by the attending physician by patient evaluation. For example, improvements in symptoms and sense of well-being may be evaluated by, but not limited to, CeD PRO, GSRS, CeD GSRS, BSFS, General Well-Being Question, SF12V2, CeD-QOL, CGA, ICDSQ and CDSD scores.

In various embodiments, administration of IMU-856 or a pharmaceutically acceptable salt or a solvate thereof results in a reduction of the CDSD score, that is, administration of IMU-856 results in a reduction in symptoms as measured by a change from baseline in CDSD score. For example, administration of IMU-856 may reduce the CDSD score by at least about 20%, at least about 30%, at least about 40%, at least about 50%, at least about 60%, at least about 70%, at least about 75%, at least about 80%, at least about 85%, at least about 90%, at least about 95%, or at least about 100%.

In various embodiments, administration of IMU-856 or a pharmaceutically acceptable salt or a solvate thereof results in a reduction of the CDSD abdominal pain score. In an embodiment, administration of IMU-856 results in a reduction in symptoms as measured by a change from baseline in CDSD abdominal pain score. For example, administration of IMU-856 may reduce the CDSD abdominal pain score by at least about 20%, at least about 30%, at least about 40%, at least about 50%, at least about 60%, at least about 70%, at least about 75%, at least about 80%, at least about 85%, at least about 90%, at least about 95%, or at least about 100%.

In various embodiments, administration of IMU-856 or a pharmaceutically acceptable salt or a solvate thereof results in a reduction of the CDSD diarrhea score. In an embodiment, administration of IMU-856 results in a reduction in symptoms as measured by a change from baseline in CDSD diarrhea score. For example, administration of IMU-856 may reduce the CDSD diarrhea score by at least about 20%, at least about 30%, at least about 40%, at least about 50%, at least about 60%, at least about 70%, at least about 75%, at least about 80%, at least about 85%, at least about 90%, at least about 95%, or at least about 100%.

In various embodiments, administration of IMU-856 or a pharmaceutically acceptable salt or a solvate thereof results in a reduction of the CDSD bloating score. In an embodiment, administration of IMU-856 results in a reduction in symptoms as measured by a change from baseline in CDSD bloating score. For example, administration of IMU-856 may reduce the CDSD bloating score by at least about 20%, at least about 30%, at least about 40%, at least about 50%, at least about 60%, at least about 70%, at least about 75%, at least about 80%, at least about 85%, at least about 90%, at least about 95%, or at least about 100%.

In various embodiments, administration of IMU-856 or a pharmaceutically acceptable salt or a solvate thereof results in a reduction of the CDSD nausea score. In an embodiment, administration of IMU-856 results in a reduction in symptoms as measured by a change from baseline in CDSD nausea score. For example, administration of IMU-856 may reduce the CDSD nausea score by at least about 20%, at least about 30%, at least about 40%, at least about 50%, at least about 60%, at least about 70%, at least about 75%, at least about 80%, at least about 85%, at least about 90%, at least about 95%, or at least about 100%.

In various embodiments, administration of IMU-856 or a pharmaceutically acceptable salt or a solvate thereof results in a reduction of the CDSD tiredness score. In an embodiment, administration of IMU-856 results in a reduction in symptoms as measured by a change from baseline in CDSD tiredness score. For example, administration of IMU-856 may reduce the CDSD tiredness score by at least about 20%, at least about 30%, at least about 40%, at least about 50%, at least about 60%, at least about 70%, at least about 75%, at least about 80%, at least about 85%, at least about 90%, at least about 95%, or at least about 100%.

In various embodiments, administration of IMU-856 or a pharmaceutically acceptable salt or a solvate thereof results in an improvement of symptoms and well-being in the celiac disease patient as measured by the average on-treatment General Well-Being Question score.

In various embodiments, administration of IMU-856 or a pharmaceutically acceptable salt or a solvate thereof results in an improvement of symptoms and well-being in the celiac disease patient as measured by the SF12V2 Questionnaire.

In various embodiments, administration of IMU-856 or a pharmaceutically acceptable salt or a solvate thereof results in an improvement of symptoms and well-being in the celiac disease patient as measured by the Celiac Disease-Quality of Life Questionnaire (CeD-QoL).

In various embodiments, administration of IMU-856 or a pharmaceutically acceptable salt or a solvate thereof results in an improvement of symptoms and well-being in the celiac disease patient as measured by the Clinician Global Assessment of Disease Activity (CGA).

IMU-856 or a pharmaceutically acceptable salt or a solvate thereof can be administered in unit dosage forms (e.g., tablets or capsules). As shown herein, subjects or patients experiencing substantial celiac disease symptoms, as well as patients identified as being non-responsive to GFD or having refractory celiac disease, can show considerable improvement even on low doses of IMU-856. For example, IMU-856 or a pharmaceutically acceptable salt or a solvate thereof can be administered from about 20 mg to about 800 mg, or from about 80 mg to about 320 mg, from about 60 mg to about 240 mg, or from about 60 mg to about 160 mg. Exemplary unit doses include about 20 mg, about 40 mg, about 60 mg, about 80 mg, about 100 mg, about 120 mg, about 150 mg, about 160 mg, about 200 mg, about 240 mg, about 280 mg, about 300 mg, about 320 mg, about 400 mg, about 480 mg, about 500 mg, or about 600 mg. The amount is adjusted to IMU-856 free base in case of a salt and/or solvate.

IMU-856 or a pharmaceutically acceptable salt or a solvate thereof can be administered in unit dosage forms (e.g., tablets or capsules). As shown herein, subjects or patients experiencing substantial celiac disease symptoms, as well as patients identified as being non-responsive to GFD or having refractory celiac disease, can show considerable improvement even on low doses of IMU-856. For example, IMU-856 or a pharmaceutically acceptable salt or a solvate thereof can be administered from about 34 μmol to about 1370 μmol, or from about 137 μmol to about 546 μmol, from about 137 μmol to about 411 μmol, or from about 137 μmol to about 273 μmol. Exemplary unit doses include about 34 μmol, about 68 μmol, about 102 μmol, about 137 μmol, about 171 μmol, about 204 μmol, about 255 μmol, about 273 μmol, about 342 μmol, about 478 μmol, about 513 μmol, about 546 μmol, about 684 μmol, about 819 μmol, about 855 μmol, about 1026 μmol.

In various embodiments, treating the gastrointestinal disease resulted in a plasma trough level in the subject or patient, which is above 20 ng/ml of IMU-856 at a steady state. For example, administration of IMU-856 may result in a plasma trough level above 20 ng/ml, above 40 ng/ml, above 60 ng/ml, above 80 ng/ml, above 100 ng/ml, above 120 ng/ml, above 140 ng/ml, above 160 ng/ml, above 180 ng/mL, above 200 ng/ml, above 240 ng/ml, above 280 ng/ml, above 320 ng/ml, above 400 ng/ml, or above 500 ng/ml.

In various embodiments, treating the gastrointestinal disease resulted in a plasma trough level in the subject or patient, which is above 20 ng/ml of IMU-856 at a steady state. For example, administration of IMU-856 may result in a plasma trough level range from about 20 ng/mL to 1000 ng/ml, from about 40 ng/ml to 500 ng/ml, from about 60 ng/ml to 320 ng/ml, from about 60 ng/ml to 200 ng/mL, from about 60 ng/ml to 200 ng/ml, from about 160 ng/ml to 320 ng/ml, from about 60 ng/ml to 90 ng/ml, from about 70 ng/ml to 85 ng/ml, from about 140 ng/mL to 200 ng/ml, or from about 150 ng/ml to 190 ng/ml.

In various embodiments, the steady state is achieved is achieved between about 3 to 14 days, about 5 to 10 days, or about 8 days after periodic or regular administration to a optionally once-a-day dosing of the drug, therapeutic combination or pharmaceutical dosage form.

In various embodiments, the plasma concentration for the drug, therapeutic combination or pharmaceutical dosage form is: (a) the trough level or trough concentration (C_{trough}), or the lowest concentration reached by the drug, therapeutic combination or pharmaceutical dosage form before a second or next dose is administered, or (b) determined from blood samples taken between about 2 hours to 24 hours, or 4 to 12 hours, or 1, 2, 3, 4, 5, 6, 7, 8, 9, or 10 or more hours, after the last dose or administration of the drug, therapeutic combination or pharmaceutical dosage form.

In various embodiments, treating the gastrointestinal disease comprises favorably altering the level of the Villous height: Crypt depth ratio. In embodiments, treating the gastrointestinal disease comprises increasing the Villous height: Crypt depth ratio. For example, the Villous height: Crypt depth ratio is determined via a biopsy. For example, administration of IMU-856 may improve the Villous height: Crypt depth ratio by at least about 20%, at least about 30%, at least about 40%, at least about 50%, at least about 60%, at least about 70%, at least about 75%, at least about 80%, at least about 85%, at least about 90%, at least about 95%, or at least about 100% compared to untreated patients or compared to before the start of treatment.

In various embodiments, treating the gastrointestinal disease comprises favorably altering the level of the Villous height. In embodiments, treating the gastrointestinal disease comprises increasing the Villous height. For example, the Villous height is determined via a biopsy. For example, administration of IMU-856 may improve the Villous height by at least about 20%, at least about 30%, at least about 40%, at least about 50%, at least about 60%, at least about 70%, at least about 75%, at least about 80%, at least about 85%, at least about 90%, at least about 95%, or at least about 100% compared to untreated patients or compared to before the start of treatment.

In various embodiments, treating the gastrointestinal disease comprises favorably altering the level of one or more biomarkers. In embodiments, treating the gastrointestinal disease comprises reducing the level of IL-2. For example, the level of biomarker IL-2 is determined from serum obtained from the subject. For example, administration of IMU-856 may reduce the level of IL-2 by at least about 20%, at least about 30%, at least about 40%, at least about 50%, at least about 60%, at least about 70%, at least about 75%, at least about 80%, at least about 85%, at least about 90%, at least about 95%, or at least about 100% compared to untreated patients or compared to before the start of treatment. In embodiments, treating the gastrointestinal disease comprises enhancing the level of citrulline. For example, the level of biomarker citrulline is determined from plasma obtained from the subject. For example, administration of IMU-856 may improve the level of citrulline by at least about 20%, at least about 30%, at least about 40%, at least about 50%, at least about 60%, at least about 70%, at least about 75%, at least about 80%, at least about 85%, at least about 90%, at least about 95%, or at least about 100%.

In various embodiments, treating the gastrointestinal disease comprises improving red blood cell function. In embodiments, the improvement red blood cell function is determined via the mean corpuscular hemoglobin concentration (MCHC). For example, the MCHC is determined from serum obtained from the subject. For example, administration of IMU-856 may improve the MCHC by at least about 20%, at least about 30%, at least about 40%, at least about 50%, at least about 60%, at least about 70%, at least about 75%, at least about 80%, at least about 85%, at least about 90%, at least about 95%, or at least about 100%.

In one embodiment, treating the gastrointestinal disease herein comprises enhancing nutrient absorption. In embodiments the nutrient is Vitamin B12. For example, the level of Vitamin B12 is determined from serum obtained from the subject. For example, administration of IMU-856 may improve level of Vitamin B12 by at least about 20%, at least about 30%, at least about 40%, at least about 50%, at least about 60%, at least about 70%, at least about 75%, at least about 80%, at least about 85%, at least about 90%, at least about 95%, or at least about 100%. In embodiments the nutrient is zinc. For example, the level of zinc is determined from serum obtained from the subject. For example, administration of IMU-856 may improve level of zinc by at least about 20%, at least about 30%, at least about 40%, at least about 50%, at least about 60%, at least about 70%, at least about 75%, at least about 80%, at least about 85%, at least about 90%, at least about 95%, or at least about 100%. In embodiments the nutrient is ferritin. For example, the level of ferritin is determined from serum obtained from the subject. For example, administration of IMU-856 may improve level of ferritin by at least about 5%, at least about 10%, at least about 20%, at least about 30%, at least about 40%, at least about 50%, at least about 60%, at least about 70%, at least about 75%, at least about 80%, at least about 85%, at least about 90%, at least about 95%, or at least about 100%.

In accordance with certain embodiments of the invention, IMU-856 or a pharmaceutically acceptable salt or a solvate thereof is administered once or more than once daily. For example, IMU-856 or a pharmaceutically acceptable salt or a solvate thereof may be administered once daily, twice daily, or three times daily. In some embodiments, IMU-856 is administered prior to meals, simultaneously with meals, or after meals, to reduce the effects of gluten exposure. In an embodiment, IMU-856 is administered prior to meals, such as about 15 minutes prior to meals. In such embodiments, IMU-856 is administered about 2 hours, about 90 minutes, about 60 minutes, about 55 minutes, about 45 minutes, about 40 minutes, about 35 minutes, about 30 minutes, about 25 minutes, about 20 minutes, about 15 minutes, about 10 minutes, about 9 minutes, about 8 minutes, about 7 minutes, about 6 minutes, about 5 minutes, about 4 minutes, about 3 minutes, about 2 minutes, or about 1 minute prior to meals.

In another embodiment, IMU-856 or a pharmaceutically acceptable salt or a solvate thereof is administered after meals. In such embodiments, IMU-856 is administered about 2 hours, about 90 minutes, about 60 minutes, about 55 minutes, about 45 minutes, about 40 minutes, about 35 minutes, about 30 minutes, about 25 minutes, about 20 minutes, about 15 minutes, about 10 minutes, about 9 minutes, about 8 minutes, about 7 minutes, about 6 minutes, about 5 minutes, about 4 minutes, about 3 minutes, about 2 minutes, or about 1 minute after meals.

In some embodiments, IMU-856 or a pharmaceutically acceptable salt or a solvate thereof may be administered for a prolonged period. Continuous IMU-856 regimens can exhibit improving symptoms over time. For example, IMU-856 may be administered as described herein for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, or at least about 26 weeks. For example, IMU-856 may be administered for at least about 8 weeks, at least about 9 weeks, at least about 10 weeks, at least about 11 weeks, or at least about 12 weeks. In some embodiments, the IMU-856 is administered for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. For example, IMU-856 is administered for at least about 6 months. In some embodiments, IMU-856 may be administered for at least about 1 year, at least about 2 years, at least about 3 years, at least about 4 years, or at least about 5 years. For example, IMU-856 may be administered for at least about 1 year.

In some embodiments, the pharmaceutical composition containing IMU-856 or a pharmaceutically acceptable salt or a solvate thereof may be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); intravenously; subcutaneously; transdermally (for example as a patch applied to the skin); and topically (for example, as a cream, ointment or spray applied to the skin). The compound may also be formulated for inhalation. In a preferred embodiment, the route of administration is oral.

In some embodiments, IMU-856 or a pharmaceutically acceptable salt or a solvate thereof compositions are administered to a subject by contacting the mucosal tissues of the gastrointestinal tract. For example, IMU-856 may be formulated for delivery to one or more of the small intestine and large intestine. By targeting release of IMU-856 in the affected region(s) (e.g., duodenum, jejunum and ileum, colon transversum, colon descendens, colon ascendens, colon sigmoidenum and cecum), tight junction integrity at any portion of the GI tract can be improved.

In some embodiments, such as for patients having short bowel syndrome, IMU-856 or a pharmaceutically acceptable salt or a solvate thereof compositions are administered to a subject intravenously.

In various embodiments, the pharmaceutical composition may be formulated to have a delayed-release profile, i.e. not immediately release the active ingredient(s) upon ingestion; rather, postponement of the release of the active ingredient(s) until the composition is lower in the gastrointestinal tract; for example, for release in the small intestine (e.g., one or more of duodenum, jejunum, ileum) or the large intestine (e.g., one or more of cecum, ascending, transverse, descending or sigmoid portions of the colon).

In some embodiments, such as for patients having non-responsive or refractory celiac disease or IBS, the patient may receive adjunct therapy, which in some embodiments is synergistic with IMU-856 treatment. In some embodiments, the additional therapeutic agent is an anti-inflammatory agent such as steroidal anti-inflammatory agents or nonsteroidal anti-inflammatory agents (NSAIDs). Steroids, particularly the adrenal corticosteroids and their synthetic analogues, are well known in the art. Examples of corticosteroids include, without limitation, hydroxyltriamcinolone, alpha-methyl dexamethasone, beta-methyl betamethasone, beclomethasone dipropionate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone and budesonide. NSAIDs that may be used in the present invention, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone and indomethacin.

In an embodiment, the additional therapeutic agent is an immunosuppressive agent such as azathioprine, cyclosporin, infliximab and alemtuzumab.

In some embodiments, the additional therapeutic agent is an antidiarrheal agent. Antidiarrheal agents suitable for use in the present invention include, but are not limited to, DPP-IV inhibitors, natural opioids, such as tincture of opium, paregoric, and codeine, synthetic opioids, such as diphenoxylate, difenoxin and loperamide, bismuth subsalicylate, lanreotide, vapreotide and octreotide, motiln antagonists, COX2 inhibitors like celecoxib, glutamine and traditional antidiarrheal remedies, such as kaolin, pectin, berberine and muscarinic agents.

In some embodiments, the additional therapeutic agent is an antibacterial agent such as an antibiotic. Antibiotics suitable for use in the present invention include, but are not limited to, cephalosporin antibiotics (cefalexin, cefuroxime, cefadroxil, cefazolin, cefalotin, cefaclor, cefamandole, cefoxitin, cefprozil, and ceftobiprole); fluoroquinolone antibiotics (ciprofloxacin, levofloxacin, moxifloxacin, gatifloxacin, and norfloxacin); tetracycline antibiotics (tetracycline, minocycline, oxytetracycline, and doxycycline); penicillin antibiotics (amoxicillin, ampicillin, phenoxymethylpenicillin, dicloxacillin, carbenicillin, vancomycin, and meticillin); monobactam antibiotics (aztreonam); and carbapenem antibiotics (ertapenem, doripenem, imipenem/cilastatin, and meropenem).

In some embodiments, the additional therapeutic agent is a probiotic. Probiotics suitable for use in the present invention include, but are not limited to, Saccharomyces boulardii; Lactobacillus rhamnosus GG; Lactobacillus plantarum 299v, Clostridium butyricum M588; Clostridium difficile VP20621 (non-toxigenic C. difficile strain); combination of Lactobacillus casei, Lactobacillus acidophilus (Bio-K+CL1285); combination of Lactobacillus casei, Lactobacillus bulgaricus, Streptococcus thermophilus (Actimel); combination of Lactobacillus acidophilus, Bifidobacterium bifidum (Florajen3); combination of Lactobacillus acidophilus, Lactobacillus bulgaricus delbrueckii subsp. bulgaricus, Lactobacillus bulgaricus casei, Lactobacillus bulgaricus plantarum, Bifidobacterium longum, Bifidobacterium infantis, Bifidobacterium breve, and Streptococcus salivarius subsp. thermophilus.

In some embodiments, IMU-856 or a pharmaceutically acceptable salt or a solvate thereof can be administered alone or in combination with additional therapeutic agents for adjunct therapy selected from
(a) a dihydroorotate dehydrogenase inhibitor, preferably vidofludimus calcium;
(b) a compound that modifies gluten or neutralizes gluten, e.g. latiglutenase, zamaglutenase, AGY 010 or GliadinX®;
(c) a transglutaminase 2 inhibitor, e.g. ZED1227/TAK-227 or GSK3915393;
(d) a compound suitable for tolerance inducing strategy, e.g. TAK-101, TPM502 or KAN-101;
(e) a compound targeting IL-15, e.g. ordesekimab, CALY-002, TEV-53408 or EQ102;
(f) an inhibitor of T-cell activation, e.g. DONQ52;
(g) a modulator of lymphocyte trafficking, e.g. PTG-100;
(h) a Janus Kinase (JAK) inhibitor, e.g. ritlecitinib or tofacitinib;
(i) a tight junction modulator, e.g. larazotide acetate; and
(j) vitamins and dietary supplements.

### Definitions

The term “IMU-856” relates to the orally bioavailable SIRT6 modulator 6-[trans-4-[4-[trans-4-[4-methyl-5-[[[4-(trifluoromethyl)-2-pyridinyl]oxy]methyl]-4H-1,2,4-triazol-3-yl]cyclohexyl]-1H-pyrazol-1-yl]cyclohexyl]-1-oxa-6-azaspiro[3.3]heptane (CAS registry number: 2303528-97-2) with the following chemical formula:

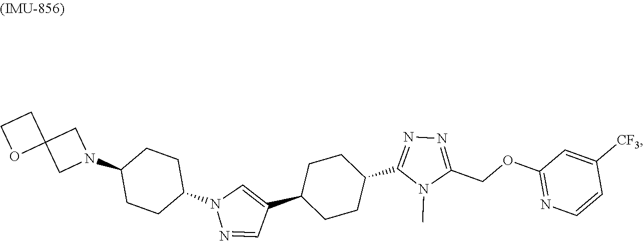 [SMILES: Cn1c(COc2cc(C(F)(F)F)ccn2)nnc1[C@H]1CC[C@H](c2cnn([C@H]3CC[C@H](N4CC5(CCO5)C4)CC3)c2)CC1]

or a pharmaceutically acceptable salt and/or solvate, in particular hydrate, thereof.

The term “pharmaceutically acceptable salts” refers to salts prepared from pharmaceutically acceptable non-toxic acids, including inorganic acids and organic acids. Thus, the compound of the present disclosure contains a basic group and can be used according to the disclosure, for example, as hydrochloride, aspartate, glutamate, L-tatrate, malonate, fumarate, citrate, malate, maleate, lactate, gluconate, benzoate, succinate, acetate, phosphate, sulfate, napsylate, besylate, tosylate or mesylate salt. The respective salts can be obtained by customary methods which are known to the person skilled in the art like, for example, by contacting these with an organic or inorganic acid in a solvent or dispersant, or by anion exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts.

“Pharmaceutically acceptable” means suitable for use in a human subject.

The term “solvate” refers to a crystalline form of a molecule that further comprises molecules of a solvent or solvents incorporated into the crystalline lattice structure. Thus, the compounds of the present disclosure may be present in the form of solvates, such as those which include as solvate water, or pharmaceutically acceptable solvates, such as alcohols, in particular ethanol. A stoichiometric or non-stoichiometric amount of solvent is bound by non-covalent intermolecular forces. When the solvent is water, the “solvate” is a “hydrate.” It is understood, that a “pharmaceutically acceptable salts” can in addition optionally contain a “solvate”.

The term “polymorph” as used herein refers to a crystalline form of a compound or a salt, or a solvate, in particular hydrate, thereof or a solvate, in particular a hydrate, of a salt thereof, in a particular crystal packing arrangement. All polymorphs have the same elemental composition. The term “crystalline” as used herein, refers to a solid-state form which consists of orderly arrangement of structural units. Different crystalline forms of the same compound, or a salt, or a solvate, in particular a hydrate, thereof or a solvate, in particular a hydrate, of a salt thereof, arise from different packing of the molecules in the solid state, which results in different crystal symmetries and/or unit cell parameter. Different crystalline forms usually have different X-ray diffraction patterns, infrared spectra, melting points, density, hardness, crystal shape, optical and electrical properties, stability, and solubility.

“A week” preferably refers to a period of time of or about 5, about 6 or about 7 days. It may be about 5-8 days.

“A month” preferably refers to a period of time of or about 28, about 29, about 30 or about 31 days. It may be about 26-33 days.

The term “treating” or “treatment” means an alleviation of symptoms associated with a disease, disorder or condition, or halt of further progression or worsening of those symptoms. Depending on the disease and condition of the subject, the term “treatment” as used herein may include one or more of curative, palliative and prophylactic treatment. Treatment can also include administering a pharmaceutical formulation of the present invention in combination with other therapies.

The term “ameliorating” means slowing, arresting or reducing the development of the disease, or at least one of the clinical symptoms thereof. In addition, those term refer to improving at least one physical parameter including those which may not be discernible by the patient and also to beneficially modulating the disease or disorder, either physically (e.g., stabilization of a discernible symptom), physiologically (e.g., stabilization of a physical parameter) or both.

The term “prevention” means that by taking IMU-856 the development of the disease or at least one of its clinical symptoms does not occur at all or is slowed down.

The term “prophylaxis” refers to the preventive treatment or measures taken to protect against the onset of a disease or infection. This could include the use of medications or other interventions aimed at reducing the risk of contracting a specific illness.

“Daily dose” preferably refers to the total dose of IMU-856 or a pharmaceutically acceptable salt or a solvate thereof, preferably orally administered to the subject or patient each day of administration. The daily dose can be reached through a single or several administrations per day, such as for example once a day, twice a day or three times a day. Preferably, it is reached or achieved by single administration per day, preferably consisting of one or more tablets or capsules, preferably tablets or capsules as described herein.

As used herein, the term “effective amount” includes a dosage sufficient to produce a desired result with respect to the indicated disorder, condition, or mental state. The desired result may comprise a subjective or objective improvement in the recipient of the dosage.

As used herein, the term “administering” includes activities associated with providing a patient an amount of IMU-856 or a pharmaceutically acceptable salt or a solvate thereof. Administering includes providing unit dosages of compositions set forth herein to a patient in need thereof. Administering includes providing effective amounts of compounds, e.g. IMU-856 or a pharmaceutically acceptable salt or a solvate thereof, for a specified period of time, e.g. for about 6, 9, 12, 15 or more months, or about 1, 2, 3, 4, 5 or more years.

As used herein, the term “patient” or “subject” refers to refers to a living mammalian organism. In one embodiment the patient is a human subject.

As used herein, the term “administered as adjunct therapy” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. In some instances, the co-administered compounds are administered by the same route. In other instances, the co-administered compounds are administered via different routes. For example, one compound can be administered orally, and the other compound can be administered, e.g. sequentially or simultaneously, via intravenous or intraperitoneal injection.

The term “about” as used herein with respect to numbers, figures, ranges and/or amounts is preferably meant to mean “circa” and/or “approximately”. The meaning of those terms is well known in the art and preferably includes a variance, deviation and/or variability of the respective number, figure, range and/or amount of plus/minus 15% and especially of plus/minus 10%.

Compositions may further comprise one or more pharmaceutically acceptable additional ingredient(s) such as alum, stabilizers, antimicrobial agents, buffers, coloring agents, flavoring agents, adjuvants, and the like.

Compositions may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, acacia, gelatine, sorbitol, tragacanth, mucilage of starch and polyvinylpyrrolidone. Fillers include, but are not limited to, lactose, sugar, microcrystalline cellulose, maize starch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate). Tablets may be coated according to methods well known in the art.

Compositions may also be liquid formulations including, but not limited to, aqueous or oily suspensions, solutions, emulsions, syrups, and elixirs. The compositions may also be formulated as a dry product for constitution with water or other suitable vehicle before use, such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatine, hydroxyethylcellulose, carboxymethyl cellulose, aluminium stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

### EXAMPLES

### Example 1A: Content of Different Doses of IMU-856

As described above, IMU-856, can be dosed as free base or as a pharmaceutically acceptable salt and/or solvate thereof. The preferred polymorph of IMU-856 (as free base) is described in WO2019/054427, i.e., is characterized by an X-ray powder diffraction pattern having characteristic peaks at 2 theta (±0.2°) of 13.2°, 15.8°, 16.5°, 17.8°, 18.1°, 20.3°, 20.8°, 21.4° and 27.9°. The quantity stated in the embodiments and claims relates to the amount of active moiety, i.e., free base. For optional salt and/or solvate the amount has to be adjusted. In the following table the amount (in mg) of active moiety of the compound is converted into μmol.

| | μmol IMU-856 |
|---|---|
| mg IMU-856 | (free base) |
| 320 | 546 |
| 240 | 411 |
| 160 | 273 |
| 80 | 137 |
| 40 | 68 |
| 20 | 34 |

### Example 2: In Vitro Intestinal Barrier Function Model

CaCo2 cell line is derived from human epithelial cells from the colon. They are frequently used to mimic intestinal barrier function to either look at permeability of drugs or to look at is functional resistance. For the present experiment, the CaCo2 cells were used to mimic the loss of its barrier function upon challenging the cells with 2,4,6-trinitrobenzene sulfonic acid (TNBS) and investigate the effect of IMU-856 on this by measuring the electrical transepithelial resistance (TEER). TNBS is a chemical that in in vivo models is often used to induce a disease that shows similarity's with Crohn's disease in humans. For this CaCo2 cells, cultivated in a 6-well trans well plate on top of the membrane that has small pores that prevent the cells from going through but allow molecules and nutrients to pass. After 3 weeks, when the TEER is stable, the monolayer is challenged with 0.25% TNBS stimulation for 4 h. Then, cells were washed and fresh medium with vehicle or IMU-856 was given. TEER was measured before and after challenge at different time points. For correct measurement, the shorter electrode is inserted into the transwell chamber (without touching the cells), and the longer electrode is inserted in the outer well.

The results show a higher TEER and a higher recovery rate for cells treated with IMU-856 compared to vehicle control. This indicate that treatment with IMU-856 induces a tightening of the barrier and that therefore, IMU-856 can be active in diseases that show a reduced intestinal barrier function like IBD, IBS and CelD.

### Example 3: Therapeutic Dextran Sodium Sulfate (DSS)-Induced Colitis Mouse Model

For this model, which is an often-used in vivo model for IBD, male C57BL/6 mice were treated with 2.8% DSS in drinking water for 5 days. Treatment with either vehicle (PEG400), 60 mg/kg sulfasalazine (positive control) or 1 mg/kg IMU-856 was given at disease onset (day 4 after start of DSS treatment) per oral gavage once daily for 5 days. Diarrhea score (normal consistency; soft; pasty, unshaped, not sticking to anus; diarrhea, sticking to anus; diarrhea with macroscopic bleeding) was assessed daily.

At the end of the study, mice were sacrificed and the colon was collected. Colon histology score consisting of an architecture score (0-3) and an infiltration score (0-3) was assessed based on hematoxylin and eosin-stained tissues slices.

This model showed that drinking of DSS induced an upregulation of the diarrhea score and this was also reflected in an increased histological score. The positive control showed a reduction in the diarrhea score while no improvement on the histology score was observed. IMU-856 also showed an improvement of the diarrhea score which was similar or even better than the positive control. In addition, IMU-856 was able to also show a reduction in the histology score.

These results show that IMU-856 has activity in the DSS-colitis model, which indicates its potential activity in human diseases in which a defective intestinal barrier function and intestinal inflammation play an important role such as UC, CD, CelD, IBS or IBS-D.

### Example 4: A Three-Part, Double-Blind, Placebo-Controlled, Phase I/Ib Study of the Safety, Tolerability and Pharmacokinetics of Single and Multiple Ascending Doses of IMU-856 in Healthy Volunteers and Patients with Celiac Disease

| | |
|---|---|
| Study | To assess the safety and tolerability of IMU-856 when |
| Objective | administered as a single oral dose in healthy adult volunteers, |
| (Primary): | as a 14-day repeat oral dose to healthy volunteers and as a 28-day |
| | repeat dose in patients with CelD. |
| Study | To assess the pharmacokinetics (PK) of IMU-856 following |
| Objectives | administration of a single dose, a 14-day repeat dose in healthy |
| (Secondary): | volunteers, and a 28-day repeat dose in patients with CelD. |
| | To assess the trough plasma concentration levels of IMU-856 |
| | in patients with CelD treated in a 28-day repeat dose schedule. |
| | To assess the safety and tolerability of administrating IMU-856 |
| | in a 28-day repeat dose schedule in patients with CelD during |
| | periods of gluten-free diet (GFD) and gluten challenge. |
| | To investigate the effect of IMU-856 on the gastrointestinal |
| | architecture and inflammation in patients with CelD during |
| | periods of GFD and gluten challenge. |
| Study | To investigate pharmacodynamic (PD) marker changes |
| Objectives | associated with administration of IMU-856 in healthy |
| (Exploratory): | volunteers and patients with CelD. |
| | To identify the metabolites of IMU-856 in plasma (Part B, |
| | Cohort 9N only). |
| | To assess treatment satisfaction and safety through patient- |
| | reported questionnaires. |
| Participant | Part A: 48 healthy volunteers will be enrolled in a total of 6 |
| Number: | cohorts. Each cohort will enrol 8 volunteers with 6 volunteers |
| | randomized to receive IMU-856 and 2 volunteers randomized |
| | to receive placebo. |
| | Part B: 24 healthy volunteers will be enrolled in a total of 3 |
| | cohorts. Each cohort will enrol 8 volunteers with 6 volunteers |
| | randomized to receive IMU-856 and 2 volunteers randomized |
| | to receive placebo. |
| | Part C: Approximately 42 patients with CelD will be enrolled in |
| | 2 cohorts. The low-dose cohort will enrol approximately 18 |
| | patients with 12 patients randomised to receive IMU-856 and 6 |
| | patients randomised to receive placebo. The high-dose cohort |
| | will enrol approximately 24 patients with 16 patients |
| | randomised to receive IMU-856 and 8 patients randomised to |
| | receive placebo. |
| Study | Part A: IMU-856 tablets (10, 20, 40, 80, 120 or 160 mg). |
| Treatment: | Part B: IMU-856 tablets (40, 80 or 160 mg). |
| | Part C: IMU-856 tablets (80 or 160 mg). |
| Comparator: | Placebo tablets (all study parts). |
| Route of | Oral. |
| Administration: | |
| Number of | Part A: One dose. |
| Doses per | Part B: There will be a total of 14 dosing days, with each daily |
| Treatment: | dose administered once a day. |
| | Part C: There will be a total of 28 dosing days. Each daily dose |
| | will be administered once a day. |
| Study | Total maximum study duration for volunteers in Part A is 44 |
| Duration: | days. |
| | Total maximum study duration for volunteers in Part B is 58 |
| | days. |
| | Total maximum study duration for patients in Part C is 84 days. |
| Study | For each volunteer enrolled in Part A, the confinement period |
| Confinement | will commence on Day −1, with dosing on Day 1 and discharge |
| periods: | on Day 4 |
| | For each volunteer enrolled in Part B, the confinement period |
| | will commence on Day −1, with once daily dosing on each Day |
| | 1-14 and discharge on Day 17. |
| | If volunteers experience any clinically significant adverse |
| | events (AEs) during the confinement period, they may remain |
| | in the clinical facility for further observation at the discretion of |
| | the Principal Investigator (PI). |
| | There will be no confinement period for patients with CelD in |
| | Part C. However, Days 1, 8 and 14 (the first day of gluten |
| | challenge) will be a full day of monitoring at the site for patients |
| | with CelD, but no overnight stay will be required. |
| Enrolment | Patients with CelD will be included in Part C of the study if they |
| Criteria for | satisfy all the following criteria. |
| Part C | 1. Adult male or female patients, 18 to 65 years of age |
| (Patients with | (inclusive) at the screening visit 1 (SV1); |
| CelD)- | 2. Patients who have been diagnosed with CelD at least 12 |
| Inclusion | months prior to randomization with initial diagnosis proven by |
| Criteria: | duodenal biopsy; |
| | 3. Successful adherence to a GFD for at least 12 months prior |
| | to randomization confirmed through a negative immunoglobulin |
| | A (IgA)-transglutaminase 2 (TG2) serology at SV1; |
| | 4. Negative urine gluten immunogenic peptide (GIP) test at |
| | SV1; In case of a positive GIP test at SV1, participants shall |
| | remain on a very strict GFD and repeat the GIP test at SV2. |
| | 5. Patients must have given written informed consent before |
| | any study-related activities are carried out and must be able to |
| | understand the full nature and purpose of the trial, including |
| | possible risks and adverse effects; |
| | 6. Body Mass Index ≥18.0 and ≤35.0 kg/m² at SV1; |
| | 7. No clinically significant abnormalities at SV1 in the following: |
| | a. Systolic blood pressure in the range of 90 to 160 mm Hg |
| | (inclusive) and diastolic blood pressure in the range of 50 to 95 |
| | mmHg (inclusive) after 5 minutes in supine position |
| | (measurements may be repeated once if the first measurement |
| | showed values outside the reference range); and/or |
| | b. Heart rate in the range of 45 to 100 beats/min (inclusive) |
| | after 5 minutes rest in supine position; and/or |
| | c. Body temperature between 35.5° C. and 37.7° C. (inclusive); |
| | and/or |
| | d. The screening 12-lead ECG must be within normal range |
| | (PR interval: 120 ms-220 ms (inclusive), QRS-Duration <120 ms, |
| | QTcF males ≤450 ms; females ≤470 ms ; and/or |
| | e. No clinically relevant findings in serum chemistry, |
| | haematology, coagulation and urinalysis examinations as |
| | judged by the Investigator; |
| | 8. Willingness to comply with a strict GFD and willingness to |
| | consume a defined amount of gluten as part of the study; |
| | 9. Have suitable venous access for blood sampling; |
| | 10. Be willing and able to comply with all study assessments |
| | and adhere to the protocol schedule and restrictions; |
| | 11. Female patients must: |
| | a. Be of non-child-bearing potential i.e., surgically sterilized |
| | (hysterectomy, bilateral salpingectomy, bilateral oophorectomy |
| | at least 6 weeks before SV1) or postmenopausal (where |
| | postmenopausal is defined as no menses for 12 months |
| | without an alternative medical cause), or |
| | b. If of child-bearing potential, must have a negative pregnancy |
| | test at SV1 (blood test) and before the first study drug |
| | administration (Day 1 urine test). They must agree not to |
| | attempt to become pregnant, must not donate ova, and must |
| | agree to: |
| | Use 2 forms of highly effective contraceptive method between |
| | signing consent, during the study, and at least 30 days after the |
| | last dose of study therapy; OR |
| | Use 1 form of highly effective contraceptive method plus an |
| | additional barrier-method of contraception between signing |
| | consent, during the study, and at least 30 days after the last |
| | dose of study therapy. Acceptable barrier methods include: |
| | i. Female diaphragm |
| | ii. Condom usage for male partner |
| | c. Women of child-bearing potential with same sex partners |
| | (abstinence from penilevaginal intercourse) are eligible when |
| | this is their preferred and usual lifestyle; |
| | 12. Male patients must agree not to donate sperm and if |
| | engaging in sexual intercourse with a female partner who could |
| | become pregnant, must agree to use a condom in addition to |
| | having the female partner use a highly effective contraceptive |
| | method between signing consent, during the study, and at least |
| | 64 days after the last dose of study therapy. |
| Exclusion | Patients with CelD will be excluded from Part C of the study if |
| Criteria: | there is evidence of any of the following at the screening visit |
| | or Day 1, prior to study drug administration: |
| | 1. Selective IgA deficiency as measured by serum IgA levels |
| | being <0.07 g/L during SV1; |
| | 2. Wheat or gluten allergy separate from CelD; |
| | 3. Signs of malabsorption as measured at SV1 by: |
| | a. Anaemia with a haemoglobin <110 g/L in women and <120 |
| | g/L in men, and/or |
| | b. Deficiency of albumin as defined by albumin <35 g/L, and/or |
| | c. Deficiency of folic acid as defined by folic acid <6.1 nmol/L; |
| | 4. History or presence of refractory CelD defined as persistent |
| | or recurrent malabsorptive symptoms and villous atrophy |
| | shown in a most recent biopsy despite strict adherence to a |
| | GFD for more than 12 months; |
| | 5. History or presence of severe complications of CelD (e.g. |
| | enteropathy associated T cell lymphoma (EATL), ulcerative |
| | jejunitis, perforation); |
| | 6. History or presence of skin manifestations of CelD like |
| | dermatitis herpetiformis at any time; |
| | 7. History or presence of neurological manifestations of CelD |
| | like ataxia or neuropathy at any time; |
| | 8. Any clinically significant concomitant disease of the intestinal |
| | tract in addition to CelD (e.g. Crohn's disease, ulcerative colitis, |
| | other form of inflammatory bowel disease, severe and |
| | therapeutically uncontrolled irritable bowel syndrome, |
| | microscopic colitis, primary sclerosing cholangitis) |
| | 9. History or presence of significant cardiovascular, pulmonary, |
| | hepatic, renal, haematological, gastrointestinal (in addition to |
| | CelD), endocrine, immunologic, dermatologic, neurological or |
| | psychiatric disease, including any acute illness or surgery |
| | within the past 3 months determined by the Investigator to be |
| | clinically relevant; |
| | 10. Known history of diabetes mellitus type 1 or type 2; 11. |
| | Known history of clinically significant liver disease (including |
| | liver cirrhosis, portal hypertension), and/or increased ALT and |
| | AST (>1.5 × upper limit of normal [ULN]) at SV1; |
| | 12. Known history of clinically significant kidney disease and/or |
| | renal function impairment, assessed as Glomerular Filtration |
| | Rate ≤60 mL/min/1.73 m² at SV1; |
| | 13. Presence of an ileostomy or colostomy or total |
| | proctocolectomy, short bowel syndrome, signs of malnutrition |
| | following gastrointestinal surgery or other major |
| | gastrointestinal surgery; |
| | 14. Current infection that requires oral and/or systemic |
| | antibiotic, antifungal, anti-parasitic or anti-viral medications, the |
| | use of oral and/or systemic antibiotic, antifungal, anti-parasitic |
| | or anti-viral medications within 4 weeks of randomization; or |
| | infections that reoccur on a regular basis, if assessed by the |
| | Investigator to be clinically significant; |
| | 15. History of chronic systemic infections including but not |
| | limited to HIV, hepatitis B or C, within 6 months before SV1; |
| | 16. Positive HBsAg, HBcAb (hepatitis B core antibody), positive |
| | HCV and/or HIV-antigenantibody (HIV-Ag/Ab) test at SV1; |
| | 17. Have or had an infection typical of an immunocompromised |
| | host and/or an infection that occurs with increased incidence in |
| | an immunocompromised host (including, but not limited to, |
| | pneumocystis jirovecii pneumonia, histoplasmosis, or |
| | coccidioidomycosis); or have a known immunodeficiency |
| | ongoing; |
| | 18. Clinically diagnosed small intestinal bacterial overgrowth, |
| | other than simple bacterial overgrowth present at initial |
| | diagnosis of CelD; |
| | 19. Receive or plan to receive probiotics during the study or |
| | ≤7 days prior to the first investigational medicinal product (IMP) |
| | administration (foods such as yogurt or kefir are acceptable); |
| | 20. History of malignant disease within the last 10 years, with |
| | the following exceptions: |
| | a. Curatively treated cervical carcinoma in situ, with no |
| | evidence of recurrence within 5 years prior to baseline may |
| | participate in the study |
| | b. Curatively treated non melanoma skin cancer with no |
| | evidence of recurrence (i.e. surgically removed basal-cell |
| | carcinoma or squamous-cell carcinoma) |
| | 21. Known, or at SV1 experienced, intolerance to |
| | lactulose/mannitol testing; |
| | 22. Any other condition or prior therapy, which, in the opinion |
| | of the Investigator, would make the patient unsuitable for this |
| | study, including unable to cooperate fully with the requirements |
| | of the study protocol or likely to be non-compliant with any |
| | study requirements; |
| | 23. Intake of corticosteroids or immunomodulators (e.g. oral or |
| | systemic glucocorticoids including oral budesonide, |
| | cyclosporine, methotrexate, anti-tumor necrosis factor-alpha |
| | therapy, anti-integrin therapy, Janus kinase inhibitors) during |
| | the past 3 months before screening biopsy sampling at SV2; |
| | 24. Intake (at least 3 times/week) of non-steroidal anti- |
| | inflammatory drugs (NSAIDs; except acetylsalicylic acid if daily |
| | dose is ≤350 mg) during the past 2 months before screening |
| | biopsy sampling at SV2; |
| | 25. Patients receiving antiplatelet therapy (i.e. acetylsalicylic |
| | acid ± combination with thienopyridines [clopidogrel, prasugrel, |
| | ticlopidine, or ticagrelor] or oral anticoagulants (i.e. warfarin, |
| | dabigatran, etexilate, rivaroxaban, apixaban); |
| | 26. Patients with known bleeding disorder or clinically relevant |
| | prolonged bleeding time as judged by the Investigator at the |
| | SV1; |
| | 27. Unwillingness to undergo upper endoscopy with biopsy |
| | during screening and during the study; |
| | 28. Known intolerance/hypersensitivity/resistance to IMP and |
| | excipients or drugs of similar chemical structure or |
| | pharmacological profile; |
| | 29. Doubt about the patient's cooperation, e.g. because of |
| | known or suspected addiction to alcohol or drugs (Cannabis |
| | excluded); |
| | 30. Positive urine drug and alcohol breain testing at SV1 or |
| | positive alcohol breath test on Day 1, prior to study drug |
| | administration; |
| | 31. Any live vaccinations within 30 days prior to study drug |
| | administration (messenger ribonucleic acid [mRNA]-based |
| | vaccinations and adenovirus vector vaccines are not |
| | considered live vaccinations); |
| | 32. Concurrent participation in any other clinical trial using an |
| | IMP or medical device, or use of any IMP within 60 days or 5× |
| | the respective half-life prior to the first study drug |
| | administration, whatever is longer; |
| | 33. Donation of blood or plasma within 30 days prior to |
| | randomization, or loss of whole blood of more than 500 mL |
| | within 30 days prior to randomization, or receipt of a blood |
| | transfusion within 1 year of the first scheduled dosing in the |
| | study; |
| | 34. Mental handicaps or disorders leading to inability to give |
| | informed consent; |
| | 35. An employee of an Investigator or Sponsor or an immediate |
| | relative of an Investigator; |
| | 36. Patients institutionalized due to judicial or administrative |
| | order. |

Study Assessments for Part C:
Clinical:
Medical history and concomitant medication use;
Evaluation of any on-study AEs;
Height and weight;
Physical examination;
Vital signs (supine systolic and diastolic blood pressure, heart rate, respiratory rate,
body temperature);
12-lead ECGs.
Blood and Urine Laboratory Assessments:
Antibodies: total IgA, IgA-TG2, IgG-deamidated gliadin peptides (DGP) (serum);
Human Leukocyte antigen (HLA) screening: Genotype HLA-DQ (blood):
DQB1*0201-DQA1*0501 (DQ2 heterodimer);
DQB1*0202 (beta chain of DQ2 with extended DR7 haplotype);
DQB1*0302 (beta chain of DQ8, there is always *0301 for A1);
Virology: HIV, HBsAg, HBcAb, and HCV antibodies;
Plasma: citrulline
Serum inflammatory markers: including but not limited to interleukin (IL)-2;
Plasma and Urine: lactulose/mannitol ratio;
Serum chemistry (malabsorption parameters): iron, ferritin, transferrin saturation,
folic acid, zinc, Vitamin B6, Vitamin B12.
Liver safety biomarker (exploratory): analysis for miR-122 will be conducted at any
time during or following completion of this study.
Kidney injury biomarker (urine) (exploratory): levels of the KIM-1 protein will be
assessed in urine to monitor for renal proximal tubule injury.
Illicit drug screen: The screen will include methamphetamines, opiates, cocaine,
phencyclidine, benzodiazepines, barbiturates, methadone, tricyclic anti-
depressants, and amphetamines;
Pregnancy test: serum human chorionic gonadotropin (hCG) for WOCBP;
Urine pregnancy test: for WOCBP (local testing at the site);
Urinary gluten immunogenic peptide (GIP) test (local testing at the site).
Safety Laboratory:
Haematology: haemoglobin, haematocrit, red blood cell (RBC) count, RBC indices,
thrombocyte count (platelets), reticulocyte count, white blood cell count with
differential (including neutrophils, eosinophils, basophils, lymphocytes and
monocytes);
Serum chemistry: sodium, potassium, chloride, bicarbonate, phosphate, calcium,
glucose, amylase, lipase, uric acid, albumin, globulins, total protein, lactate
dehydrogenase, creatine kinase, creatinine (including calculated creatinine
clearance using Cockcroft-Gault formula), GFR calculated with CKD-EPI (Chronic
Kidney Disease Epidemiology Collaboration) creatinine equation, urea, alkaline
phosphatase, ALT, AST, GGT, total bilirubin, conjugated and unconjugated
bilirubin, total cholesterol, high density lipoproteins, low density lipoproteins,
triglycerides, C-reactive protein;
Coagulation: PT, INR, aPTT (central laboratory);
Urinalysis: pH, specific gravity, protein, glucose, ketones, bilirubin, blood, nitrites,
urobilinogen, leukocyte esterase. Urine sediment microscopy will be conducted in
the instance of abnormal urinalysis findings (for all abnormal findings apart from
abnormal findings of bilirubin, urobilinogen and ketones).
Biopsy Assessments (done at the central pathology laboratory):
Quantitative-Mucosal Algorithmic Rules for Scoring Histology (Q-MARSH);
Intraepithelial lymphocyte (IEL) density (CD3 immunohistochemical [IHC] staining);
Villous height (Vh), crypt depth (Cd), and Vh/Cd ratio.
Additional assessments, depending on outcome of clinical trial (done at central
laboratory):
Blood:
Genotyping: including but not restricted to CYP3A4 and Sirtuin (SIRT) 6
Biopsy assessments:
IEL density (CD8, Ki67 IHC staining);
Lamina propria plasma cell densities (interferon regulatory factor 4 [IRF-4] IHC
staining),
mRNA expression ratio of apolipoprotein A4 (APOA4) and kib7
Safety and Tolerability:
In addition to clinical laboratory testing further specific assessments to evaluate
treatment safety include the following: AE monitoring, physical examinations, body
weight, 12-lead ECGs, vital signs.
Pharmacodynamics:
Blood (serum and plasma), urinary and tissue samples will be collected for analysis
and identification of IMU-856 induced PD marker changes.
Patient report outcomes:
Celiac Diseases Symptom Diary (CDSD) questionnaire and Impact of Celiac
Disease Symptoms Questionnaire (ICDSQ) will be determined.
Pharmacokinetics:
Blood samples for PK analysis will be collected prior to dosing and at several
timepoints post dose (at Day 8).
Study Endpoints for Part C:
Safety:
Safety endpoints include incidence, severity and causality of AEs, changes in
clinical laboratory parameters (e.g., serum chemistry, haematology, coagulation
and urinalysis parameters), body weight, physical examination findings, vital signs,
12-lead ECGs, and biopsy. Measurement of miR-122 in patients with CelD will be
included as an indicator of potential drug induced liver injury (blood, exploratory).
Measurement of KIM-1 in patients with CelD will be included as an indicator of
potential drug induced renal proximal tubule injury (urine, exploratory).
Histological and IHC assessments (e.g., Vh, Cd, Vh:Cd ratio, IELs) will be used to
assess the safety of IMU-856 administration during periods of GFD and a 15-day
gluten challenge.
Pharmacokinetics:
Plasma concentrations of IMU-856 at specified timepoints post dose will be used to
calculate PK parameters for patients with CelD. Also, plasma concentration data
(predose trough study drug concentrations) will be evaluated.
PK parameters will be calculated from the individual blood concentration-time data
of IMU-856 as described for Parts A and B (i.e. AUC_{0-inf}, Cₘₐₓ, kel, t_{1/2}, Tₘₐₓ, CL/f,
V_{d}/f, AUC₀₋ₜₗₐₛₜ, AUC₀₋₂₄) at Day 8.
Additional parameters may be calculated if deemed appropriate.
Exploratory endpoints:
Pharmacodynamics: PD marker analyses will be performed using blood, and urine
samples obtained prior to dosing (baseline) and at a number of timepoints post
dose, and will include the following:
Serological marker: IgA-TG2, IgG-DGP;
Non-invasive marker for epithelial damage: citrulline, plasma;
Non-invasive markers for inflammation: including but not limited to IL-2, serum;
Non-invasive markers for intestinal permeability: Lactulose/Mannitol ratio, urine and
plasma;
Non-invasive markers for gluten intake: urinary gluten immunogenic peptides
(GIPs);
Malabsorption parameters: iron, ferritin, transferrin saturation, folic acid, zinc,
Vitamin B6, Vitamin B12; serum.
Patient Reported Outcomes: Exploratory treatment satisfaction and safety
endpoints in patients with CelD will be evaluated based on the AE rate and changes
in the respective patient-reported questionnaires: CDSD questionnaire and ICDSQ.
Exploratory Endpoints: Additional PD marker analyses will be performed depending
on outcome of clinical trial using duodenal tissue samples obtained prior to dosing
(baseline) and at a number of timepoints post dose, and will include the following
IEL density (CD8, Ki67 IHC staining);
Lamina propria plasma cell densities (IRF-4 IHC staining);
mRNA expression ratio of APOA4 and Ki67.
Data Analyses:
Safety and Tolerability:
The number of treatment-emergent AEs (TEAEs) and serious TEAEs as well as the
number and percentage of participants with TEAEs and serious TEAEs, will be
summarized by Medical Dictionary for Regulatory Activities system organ class
(SOC) and preferred term and tabulated for each dose level. Summaries by severity
and relationship will also be presented. The percentage of participants who
discontinued treatment due to a TEAE will be summarized and tabulated for each
dose level. Symptom events that occur during the gluten challenge will be assessed
by investigator as to whether they are expected due to gluten and such expected
gluten related symptoms will not be considered AEs, unless they are unexpected in
terms of severity, duration, or appearance.
For histological and IHC assessments (e.g., Vh, Cd, Vh:Cd ratio, IELs) raw data and
change from baseline will be summarized per dose level using descriptive statistics
and presented graphically, as appropriate.
Raw data and change from baseline in vital signs, body weight, ECG and clinical
laboratory parameters will be summarized by dose level using descriptive statistics.
Abnormalities will be tabulated by treatment group showing participant counts and
percentages.
Physical examination findings will be listed.
For urine KIM-1 and plasma miR-122 biomarker levels, raw data and change from
baseline will be summarized per dose level using descriptive statistics and
presented graphically, as appropriate.
Pharmacokinetics:
Mean and individual IMU-856 plasma concentrations for each dose cohort will be
presented graphically with concentration displayed on a linear and logarithmic
scale. Plasma concentration data at each timepoint will be summarised for each
dose cohort using descriptive statistics. Pharmacokinetic parameters, including
amount of drug excreted in urine per interval (Part A only), will be determined and
summarized by dose cohort using descriptive statistics. Analyses using linear
models will be performed to assess dose proportionality (both single dose and
multiple dose), time dependence, accumulation, and attainment of steady state
(multiple dose).
Pharmacodynamics:
Pharmacodynamic markers-observed data and change from baseline-will be
summarized using descriptive statistics and graphs (mean Å} SEM) per dose cohort,
per treatment arm and time interval as appropriate.
Patient Reported Outcomes:
For Part C, CDSD and ICDSQ scores will be summarised at respective timepoints,
by treatment group for each parameter. Actual values and changes from baseline
will be presented. An analysis of covariance will be performed on mean scores and
the estimated difference between IMU-856 and placebo with 95% confidence
intervals will be calculated for low dose vs. placebo and high dose vs. placebo.
Interim analysis (Part C):
Two optional group-level unblinded interim analyses of selected safety and disease
assessment data are planned during this study for the purposes of planning further
steps in the development of IMU-856 by the Sponsor. These may occur at the
following timepoints:
1. Following the completion of the Day 29 visit for participants in Cohort 10. No study
enrollment decisions will be made based on this information (screening, enrollment
and dosing of Cohort 11 may occur in parallel with the interim analysis).
2. Following the completion of the Day 29 visit for participants in Cohort 11
Every effort will be made to restrict knowledge of group-level unblinded interim
results to the statisticians preparing the analysis and the Sponsor only. Full details
of these optional interim analyses, including details of assessments, will be
described in the statistical analysis plan.

### Results:

FIG. 1 illustrates the flow chart of part C in celiac disease (CelD) designed as proof-of-concept study with a gluten challenge trial. Part C was planned to include a well-controlled celiac disease patient population: The patients were 100% seronegative, indicating absence of ongoing substantial gluten exposure and was performed at sites in Australia and New Zealand. Part C was designed to assess safety, tolerability and pharmacokinetics of IMU-856 and measured also histologic changes, blood biomarkers, nutrient uptake and disease-related symptoms. FIG. 2 shows the patients per treatment group for Part C of the study in CelD patients.

A favorable pharmacokinetics, confirming data from Parts A+B in healthy human subjects, with fast achievement of steady state and linear pharmacokinetics with dose-proportional increase in plasma Cₘₐₓ and AUC was obtained:

Day 8

| Value (Mean) | IMU-856 80 mg | IMU-856 160 mg |
|---|---|---|
| Cₘₐₓ (ng/mL) | 354.5 | 765.6 |
| Tₘₐₓ (hr) | 1.9 | 1.5 |
| AUC_{0-t last} | 1568 | 3613 |
| (hr*ng/mL) | | |

Cₘₐₓ: maximum plasma drug concentration;
hr: hours;
Tₘₐₓ: time toreach maximum plasma concentration;
AUC: area under the curve

FIG. 3 shows the mean trough levels of IMU-856 from Part C with fast achievement of steady state and then constant exposure within the whole dosing period.

As illustrated in the following table, the distribution of Q-MARSH Histology Score at baseline shows, that approximately 60% of IMU-856 treated patients are already in FDA-required phase 2/3 population category:

| | | IMU-856 | IMU-856 | All | All |
|---|---|---|---|---|---|
| Q-MARSH | | 80 mg | 160 mg | Active | Placebo |
| N (%) | | (N = 14) | (N = 15) | (N = 29) | (N = 14) |
| M0 | | 1 (7.1) | 1 (6.7) | 2 (6.9) | 0 |
| M1 | | 1 (7.1) | 2 (13.3) | 3 (10.3) | 3 (21.4) |
| M2 | | 2 (14.3) | 5 (33.3) | 7 (24.1) | 8 (57.1) |
| Ongoing | M3a | 10 (71.4) | 6 (40.0) | 16 (55.2) | 3 (21.4) |
| Active | M3b | 0 | 1 (6.7) | 1 (3.4) | 0 |
| Celiac | M3c | 0 | 0 | 0 | 0 |
| Disease | | | | | |
| (OACD) | | | | | |

| | | | | |
|---|---|---|---|---|
| Vh: Cd (Mean) | 1.71 | 1.86 | 1.79 | 2.01 |

The patient population for the study was clinically defined as well-controlled celiac disease by seronegativity as well as by absence of signs and symptoms indicating ongoing active celiac disease (OACD). Biopsie results were not an inclusion criterium for this study. The IMU-856 treatment arms showed a higher proportion of patients having more severe disease (Q-Marsh scores of M3a or worse), for which the following is known from Stamnaes et al. (medRxiv; doi.org/10.1101/2020.05.04.20090977: A more severe baseline inflammation measured as e.g., lower Villous height: Crypt depth (Vh:Cd) ratio leads to stronger histologic and symptomatic worsening during gluten challenge.

As illustrated in FIG. 4, IMU-856 protects villous height as compared to placebo, reaching statistical significance (Wilcoxon Two-Sample Test comparison between pooled IMU-856 groups and placebo, performed as post-hoc exploratory statistical analysis. Disease Analysis Set: N=35/43 included in histology analysis set. 8 patients not included in this analysis due to early termination, all unrelated to IMP) for this objective readout which is known to be relevant to influence future medical complications of celiac disease: The data was assessed by a central pathology laboratory and blinded pathology reader. FIG. 5 illustrates, that IMU-856 shows signals of preventing histological damage (Q-MARSH) via the change in Q-MARSH score between Baseline and Day 29.

Treatment with IMU-856 also improves patients' symptoms as illustrated in FIG. 6: IMU-856 treated patients report fewer symptoms (nausea, abdominal pain or diarrhea) after first day of gluten challenge than placebo patients (at Day 14) as assessed via Celiac Disease Symptom Diary (CDSD). Fewer symptoms includes either less patients with symptoms or less severity of symptoms. IMU-856 treated patients also recover to a higher extent from bloating symptoms and from tiredness symptoms on continued treatment during gluten challenge as illustrated in FIG. 7A and FIG. 7B, respectively.

Also, biomarker response upon treatment of CelD with IMU-856 was assessed. IMU-856 dampens gluten-induced acute immune response, as measured by IL-2 release, in a dose-dependent fashion. A wide inter-individual range of IL-2 response leads to large SD, especially in the placebo group. The inter-individual variance regarding IL-2 response decreases with increasing dose levels as illustrated in FIG. 8 (N=39/43 included in IL-2 analysis set. 3 patients not included in this analysis due to early termination and 1 incomplete IL-2 sampling due to site error. Methods used for IL-2 analysis: Milliplex® Human High Sensitivity T Cell Magnetic Bead Panel, 3 plex (Luminex®) and Simoa® CorPlex™ Human IL-2 Kit (Quanterix)). Citrulline is a non-protein amino acid that is mainly produced by the enterocyte and, hence, the level of citrulline in plasma can represent the synthetic function of the enterocytes (Singh et al., J. Clin. Med. 2019: 8; 885). Plasma citrulline levels are known to be related to villous atrophy: Citrulline levels increase with gluten-free diet and with improvement of enteropathy (Fragkos et al., United Eur. Gastroenterol. J. 2018: 6; 181) and IMU-856 increased citrulline levels dose proportionally, whereas being reduced in placebo treated celiac disease patients.

It was also found that treatment of CelD with IMU-856 enhances nutrient absorption in a dose-dependent manner like Vitamin B12 (FIG. 10A) and zinc (FIG. 10B): Also, the treatment ameliorates the gluten-induced iron malabsorption, as exemplified with the respective ferritin levels (FIG. 10C). Because of improved iron absorption due to treatment with IMU-856, red blood cell function was also improved as evidenced by a higher mean corpuscular hemoglobin concentration (MCHC), which is a measure of the concentration of hemoglobin in red blood cells. Since hemoglobin is the molecule to which oxygen attaches, MCHC is a measure of the average oxygen-carrying capacity of the red blood cells in circulating the body (https://www.verywellhealth.com/mean-cell-hemoglobin-concentration-4584155).

Treatment with a suitable dose of IMU-856 shows an initial signal of protection against acute gluten-induced symptoms on first day of gluten challenge, suggesting that IMU-856 can be administered prophylactically to humans at risk of gluten intolerance (e.g., if the person is not sure whether the food contains gluten in a restaurant):

Mean change from last day before gluten challenge to
first day of gluten challenge

| | IMU-856 | IMU-856 | | |
|---|---|---|---|---|
| | 80 mg | 160 mg | All Active | All Placebo |
| | (N = 13) | (N = 13) | (N = 26) | (N = 12) |
| | Mean | Mean | Mean | Mean |
| Symptom | (SD) | (SD) | (SD) | (SD) |
| Nausea | 0.2 (0.69) | 0.7 (1.03) | 0.4 (0.90) | 0.8 (1.22) |
| Abdominal | 0.5 (0.78) | 0.4 (0.87) | 0.5 (0.81) | 0.8 (1.06) |
| pain | | | | |
| Diarrhea | 0.5 (0.97) | 0.6 (1.12) | 0.6 (1.03) | 0.8 (1.36) |

In addition, treatment with IMU-856 shows an initial signal of protection against chronic gluten-induced symptoms:

| | Mean change Day 13 to Day 14 | | | Mean change Day 14 to Day 29 | | |
|---|---|---|---|---|---|---|
| | | Bloating | Tiredness | | Bloating | Tiredness |
| | | Mean | Mean | | Mean | Mean |
| | N | (SD) | (SD) | N | (SD) | (SD) |
| IMU-856 | 13 | 0.3 | 0.2 | 9 | −0.4 | −0.1 |
| 80 mg | | (0.85) | (0.60) | | (1.01) | (0.33) |
| IMU-856 | 13 | 0.5 | 0.5 | 11 | −0.5 | −0.5 |
| 160 mg | | (0.88) | (0.66) | | (1.04) | (1.04) |
| All | 26 | 0.4 | 0.3 | 20 | −0.5 | −0.3 |
| Active | | (0.85) | (0.63) | | (1.00) | (0.80) |
| All | 12 | 0.5 | 0.8 | 8 | 0.1 | 0.1 |
| Placebo | | (0.67) | (1.14) | | (0.83) | (0.64) |

Data from four key dimensions of celiac disease pathophysiology and outcomes are summarized as follows:

Protection of gut architecture and reduction of gluten-induced intestinal damage: Over the course of the trial, the placebo group (N=11) experienced a 60.3 μm reduction in villous height in response to four weeks of treatment and two weeks of 6 g/day gluten challenge. In contrast, this reduction was only 20.9 μm and 22.5 μm for the 80 mg (N=11) and 160 mg (N=13) IMU-856 groups, respectively (p=0.04). Villi are small finger-like projections found in the lumen of the small intestine, which play a key role in the absorption of digested nutrients. Decrease in villous height is a well-recognized measure of gluten-induced damage in celiac disease and a main reason for signs and symptoms of malabsorption. Similar results were also seen for the villous height to crypt depth ratio, another common measure employed in celiac disease. Of particular interest, during the course of the trial two IMU-856 treated patients demonstrated a 1-category improvement in Q-Marsh scores despite 15 consecutive days of exposure to gluten challenge. No analogous improvement was seen with placebo treatment.

Improvement in clinical symptoms of gluten exposure: On the first day of gluten challenge, the placebo group (N=12) experienced a marked average increase in symptoms, as assessed by the Celiac Disease Symptom Diary using a 5-point scale of 0.8 for nausea, 0.8 for abdominal pain, and 0.8 diarrhea. In contrast, among the pooled IMU-856 arms (N=26), these average increases were only 0.4 for nausea, 0.5 for abdominal pain and 0.6 for diarrhea. Continued treatment with IMU-856 during gluten challenge also resulted in improvement in disease-related symptoms, such as bloating and tiredness, as well as in patient-reported ability to perform daily activities.

Dose-dependent changes in biomarker response: Following the initiation of gluten challenge on day 14, patients treated with IMU-856 exhibited a dose-dependent lessening of acute immune response, as measured by serum interleukin-2 (IL-2) release, compared with placebo. IL-2 is a well-recognized biomarker of acute gluten exposure which has been correlated by independent third parties with timing and severity of symptoms after gluten exposure. In addition, average levels of citrulline, a biomarker for the proper function of gut wall cells, were increased in both active treatment arms while decreasing levels were observed in placebo-treated patients, reflecting the protective effect of IMU-856 on the gut lining.

Correction of malabsorption-related parameters: Over the course of the trial, the placebo group (N=11) experienced a 31.0 μmol/L reduction in levels of vitamin B12. In contrast, the 80 mg (N=11) and 160 mg (N=13) IMU-856 groups experienced a 45.8 pmol/L and 74.2 μmol/L increase in vitamin B12 levels, respectively. Vitamin B12 is a nutrient essential for the formation of red blood cells, and the normal functioning of the brain and nervous system, whose absorption from the gastrointestinal tract is frequently impaired in patients with celiac disease. A similar responses to treatment was observed for zinc. Mean concentrations of ferritin decreased to a lesser extent from baseline to day 29.

### Example 5: Forced Tapering Study Design (See FIG. 12) in Steroid-Dependent Crohn's Disease (CD) and Ulcerative Colitis (UC)

| | |
|---|---|
| Main | Steroid-dependent patients with CD or UC in remission |
| Inclusion | (a) unable to taper steroids completely within 3 months |
| Criteria: | of starting steroids or-previously relapsed within |
| | 2 months after stopping steroids; (b) no IBD related |
| | co-medication allowed |
| Primary | (a) Number of patients with response to IMU-856 |
| endpoints: | ( partial response); |
| | (b) Complete response defined as steroid-free clinical |
| | remission at week 12; |
| | (c) Partial response defined as being in remission at any |
| | steroid dose equal or lower than the individual threshold |
| | dose for relapse of the individual patient; |
| | (d) The threshold dose is defined as the individual steroid |
| | dose at which a patient experienced a relapse in medical |
| | history; |
| | (e) Remission is defined as Crohn's Disease Activity |
| | index CDAI ≤150 (CD) or Clinical Activity Index |
| | CAI ≤4 (UC), while a relapse is defined as CDAI >220 |
| | (CD) or CAI >6 (UC). |
| Secondary | Secondary endpoints included CDAI/CAI at each visit, |
| endpoints: | C-reactive protein (CRP), erythrocytic sedimentation rate |
| | (ESR), calprotectin and change of prednisolone threshold |
| | dose at week 12 or at the time of relapse. |

In summary, this data provides first clinical evidence that IMU-856's ability, observed in preclinical studies, to re-establish proper gut cell renewal translates into clinical benefits for patients with celiac disease. Most importantly, the observed protection of intestinal villi from gluten-induced destruction independent of targeting celiac disease-specific disease mechanisms appears to be unique among proposed therapeutic approaches [and may allow the transfer of this concept to other gastrointestinal diseases such as ulcerative colitis, Crohn's disease or irritable bowel syndrome with diarrhea.] IMU-856 was observed to be safe and well-tolerated in this trial. There were no investigational medicinal product (IMP)-related serious or treatment-emergent adverse events nor was there any dose-dependency in adverse events. Moreover, the rates of treatment-emergent adverse events in non-disease related parameters were comparable between the active treatment groups and placebo.

### BRIEF DESCRIPTION OF THE DRAWINGS

FIG. 1 illustrates the flow chart of part C in celiac disease (CelD).

FIG. 2 shows the patients per treatment group for Part C of the study in CelD patients and reasons for early termination.

FIG. 3 shows the mean trough levels (ng/ml) of IMU-856 from Part C.

FIG. 4 illustrates that IMU-856 protects villous height as compared to placebo by showing the absolute change in villous height (μm) between Baseline and Day 29 (SD: standard deviation).

FIG. 5 shows that IMU-856 shows signals of preventing histological damage (Q-MARSH) via the change in Q-MARSH score between Baseline and Day 29. Deterioration/improvement is defined as at least a decrease/increase of 1 category in Q-MARSH score at Day 29 as compared to Baseline.

FIG. 6 shows that IMU-856 treated patients report fewer symptoms (nausea, abdominal pain or diarrhea) after first day of gluten challenge (SD: standard deviation).

FIG. 7 shows that IMU-856 treated patients also recover to a higher extent from bloating symptoms (Part A) or tiredness symptoms (Part B) on continued treatment during gluten challenge (Day 13: Last day before gluten challenge. Day 14: First day of gluten challenge. Day 29: First day after completion of gluten challenge; SD: standard deviation).

FIG. 8 illustrates the mean absolute change and maximum change in serum IL-2 (in pg/mL) from pre-gluten to 4 hours post-gluten on first day of gluten challenge (IL: interleukin; SD: standard deviation).

FIG. 9 shows that IMU-856 dampens acute IL-2 immune response on first day of gluten challenge as outlined as mean absolute change and maximum change in serum IL-2 (in pg/mL) from pre-gluten to 4 hours post-gluten on first day of gluten challenge (Number of patients: Placebo: N=13 for mean change Baseline to Day 14, N=11 for mean change Baseline to Day 29; IMU-856 80 mg: N=14 for mean change Baseline to Day 14, N=11 for mean change Baseline to Day 29; IMU-856 160 mg: N=13 for mean change Baseline to Day 14, N=13 for mean change Baseline to Day 29; SD: standard deviation).

FIG. 10 illustrates the mean change from Baseline to Day 29 for vitamin B12 (pmol/L) (Part A), for zinc (μmol/L) (Part B) and for ferritin (μg/L) (Part C).

FIG. 11 shows the mean change from Baseline to Day 29 in the mean corpuscular hemoglobin concentration (g/L) indicating an improvement in red blood cell function as a consequence of improved iron absorption due to treatment with IMU-856.

FIG. 12 illustrates the forced tapering study design in steroid-dependent Crohn's disease and ulcerative colitis.

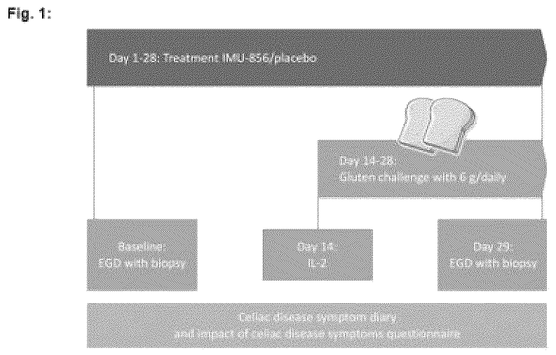

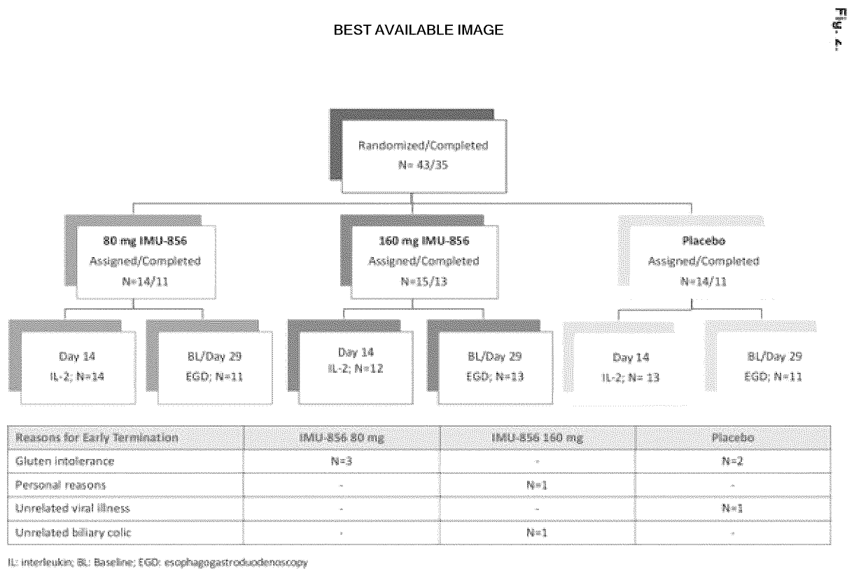

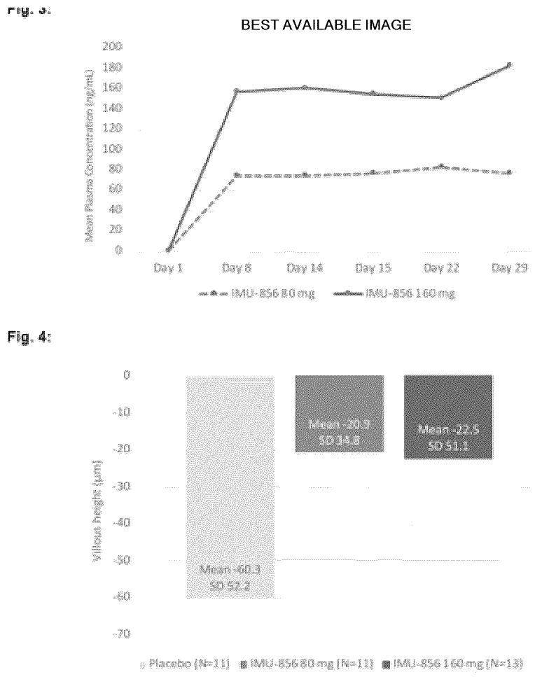

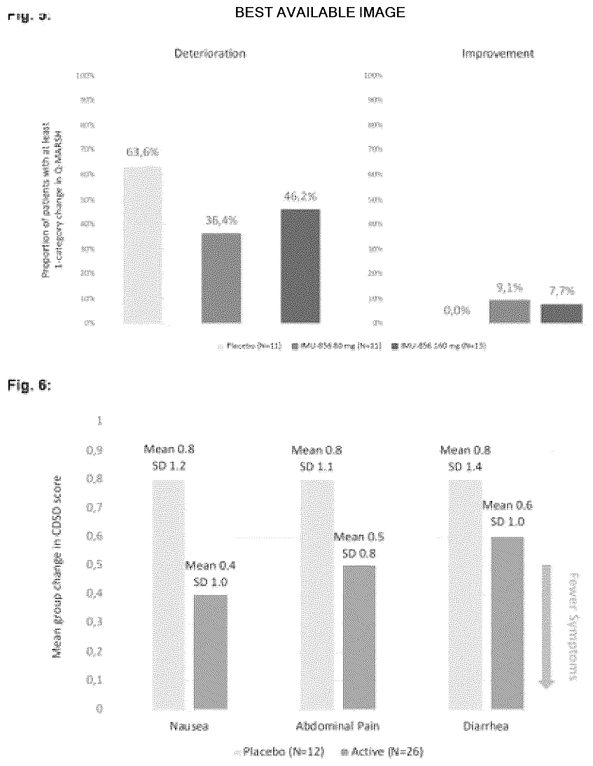

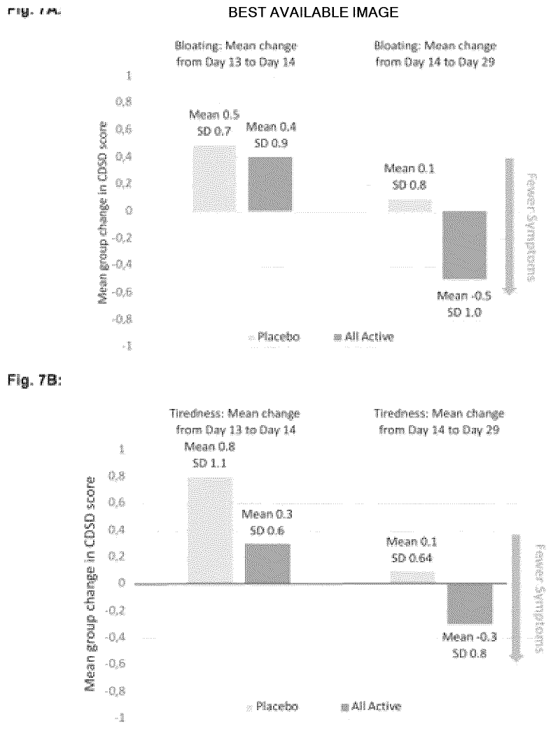

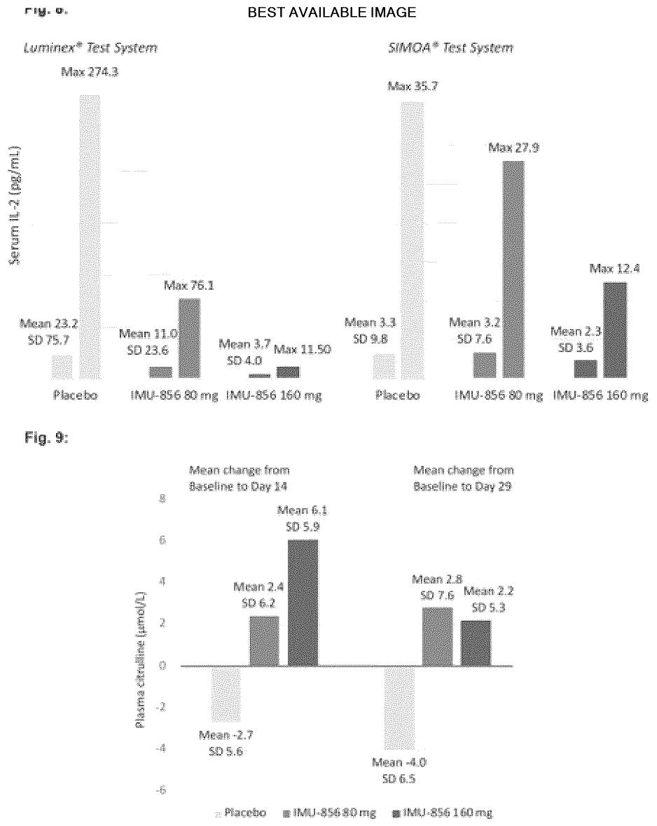

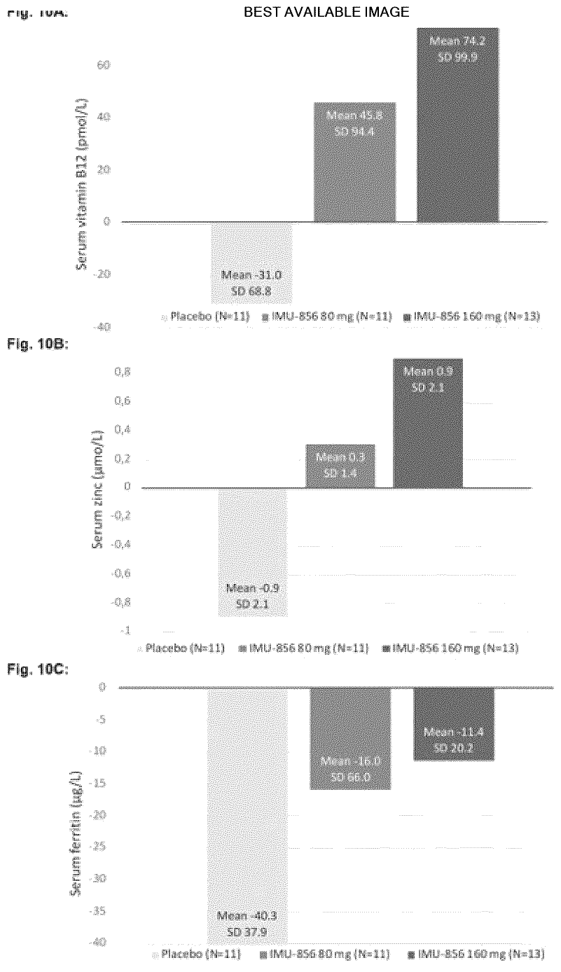

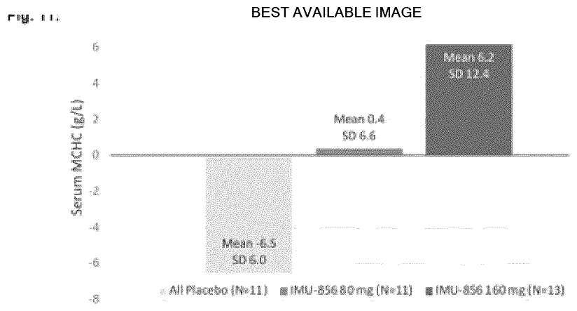

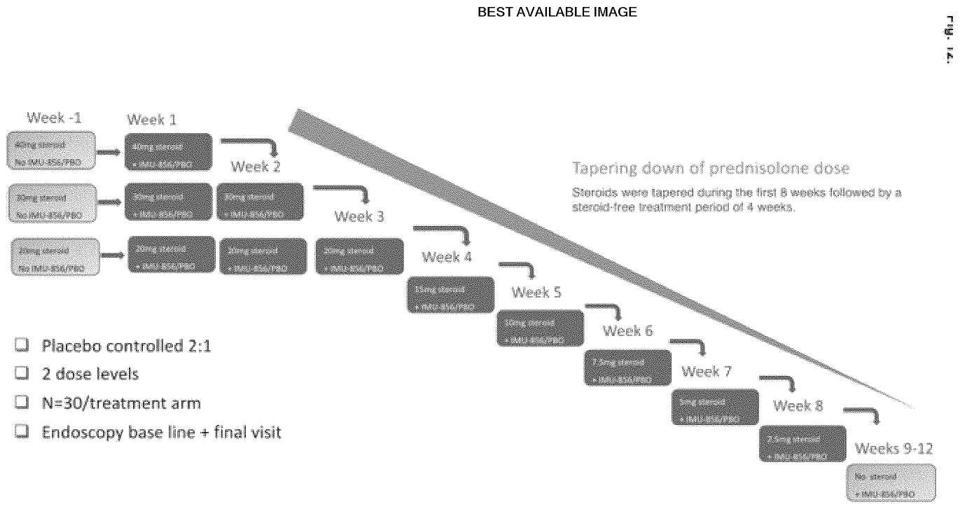

1. A compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) 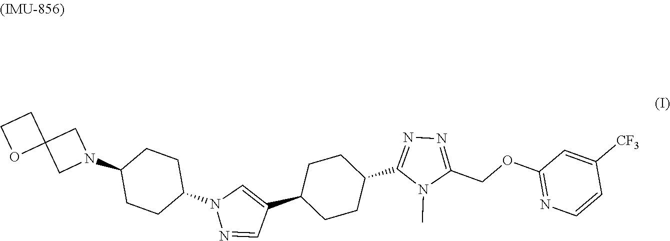 [SMILES: Cn1c(COc2cc(C(F)(F)F)ccn2)nnc1[C@H]1CC[C@H](c2cnn([C@H]3CC[C@H](N4CC5(CCO5)C4)CC3)c2)CC1]
or a pharmaceutically acceptable salt or a solvate thereof for use as a medicament for preventing, ameliorating or treating an inflammatory condition in a patient in need thereof, wherein the compound of formula (I) or a pharmaceutically acceptable salt or solvate thereof is administered to said patient in a daily dose of about 34 to about 1370 μmol.

2. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or a solvate thereof for use as a medicament for preventing, ameliorating or treating an inflammatory condition in a patient in need thereof according to claim 1, wherein said inflammatory condition is a gastrointestinal disease.

3. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or a solvate thereof for use as a medicament for preventing, ameliorating or treating an inflammatory condition in a patient in need thereof according to claim 2, wherein the gastrointestinal disease selected from celiac disease, atypical wheat allergy, gluten intolerance (e.g., non-celiac gluten sensitivity, gluten ataxia, dermatitis herpetiformis and wheat allergy), inflammatory bowel disease (e.g., Crohn's disease, ulcerative colitis or inflammatory bowel disease-associated diarrhea), irritable bowel syndrome (especially irritable bowel syndrome with diarrhea), microscopic colitis (e.g., collagenous colitis and lymphocytic colitis), short bowel syndrome and eosinophilic esophagitis.

4. The that compound is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or a solvate thereof for use as a medicament for preventing, ameliorating or treating an inflammatory condition in a patient in need thereof according to claim 3, wherein said gastrointestinal disease is a celiac disease.

5. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or a solvate thereof for use as a medicament for preventing, ameliorating or treating an inflammatory condition in a patient in need thereof according to any of claims 1 to 4, wherein said daily dose is of about 137 to about 411 μmol, preferably of about 137 to about 273 μmol.

6. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of claims 1 to 5, wherein said compound of formula (I) or a pharmaceutically acceptable salt or a solvate thereof is administered orally.

7. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of claims 1 to 6, wherein said compound of formula (I) or a pharmaceutically acceptable salt or a solvate thereof is administered once or twice daily, preferably once a day.

8. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of claims 1 to 7, wherein the compound of formula (I) is administered in a daily dose of about 80 to 240 mg, preferably of about 80 to about 160 mg.

9. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of claims 1 to 8, wherein said compound of formula (I) or a pharmaceutically acceptable salt or a solvate thereof is administered with at least one additional therapeutic target.

10. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of claims 2 to 9, wherein treating the gastrointestinal disease resulted in a plasma trough level in the subject or patient, which is above 20 ng/ml of the compound of formula (I).

11. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of claims 2 to 10, wherein treating the gastrointestinal disease comprises favorably altering the level of one or more biomarkers.

12. The compound that is 6-(trans-4-(4-(trans-4-(4-methyl-5-(((4-(trifluoromethyl)pyridin-2-yl)oxy)methyl)-4H-1,2,4-triazol-3-yl)cyclohexyl)-1H-pyrazol-1-yl)cyclohexyl)-1-oxa-6-azaspiro[3.3]heptane of formula (I) or a pharmaceutically acceptable salt or solvate thereof for use as a medicament for preventing, ameliorating or treating an inflammatory condition in a patient in need thereof according to any of claims 2 to 11, wherein treating the gastrointestinal disease comprises enhancing nutrient absorption.
